(19) United States
(12) Patent Application Publication
(10) Pub. No.: US 2023/0119120 A1
(43) Pub. Date: Apr. 20, 2023

(54) USE OF HYDROXYCINNAMIC ACID SALTS FOR STABILIZING ORGANIC MATERIALS, STABILIZED ORGANIC MATERIAL, METHOD FOR STABILIZING ORGANIC MATERIALS, SPECIFIC STABILIZERS AND STABILIZER COMPOSITIONS

(71) Applicant: FRAUNHOFER-GESELLSCHAFT ZUR FÖRDERUNG DER ANGEWANDTEN FORSCHUNG E.V., München (DE)

(72) Inventors: Rudolf PFAENDNER, Darmstadt (DE); Jannik MAYER, Darmstadt (DE); Elke METZSCH-ZILLIGEN, Darmstadt (DE)

(73) Assignee: FRAUNHOFER-GESELLSCHAFT ZUR FÖRDERUNG DER ANGEWANDTEN FORSCHUNG E.V., München (DE)

(21) Appl. No.: 17/907,038

(22) PCT Filed: Mar. 25, 2021
(86) PCT No.: PCT/EP2021/057775

§ 371 Date: Sep. 22, 2022

(30) Foreign Application Priority Data
Mar. 27, 2020 (DE) 10 2020 203 988.7

(51) Int. Cl. C08K 5/098; C08J 3/20; C08K 5/00

(52) U.S. Cl. CPC C08K 5/098; C08J 3/203; C08K 5/005; C08J 2323/12

(57) ABSTRACT

The invention relates to the use of specific hydroycinnamic acid salts for stabilizing organic materials. The invention also relates to a corresponding organic material stabilized by incorporating a specific hydroxycinnamic acid salt, and to a method for stabilizing organic materials. In addition, a specific aluminum salt of a hydroxycinnamic acid suitable as an organic stabilizer is described. The invention further relates to a stabilizer composition comprising a corresponding hydroxycinnamic acid salt.

The present invention relates to the use of specific hydroxycinnamic acid salts for stabilizing organic materials. The invention additionally relates to an organic material correspondingly stabilized by incorporating a specific hydroxycinnamic acid salt and to a method of stabilizing organic materials. In addition, a specific aluminum salt of a hydroxycinnamic acid is described that is suitable as an organic stabilizer. The present invention furthermore relates to a stabilizer composition that includes a corresponding hydroxycinnamic acid salt.

Organic materials such as plastics are subject to aging processes that ultimately result in a loss of the desired properties such as of the mechanical characteristic values. This process, called autoxidation, leads to changes in the polymer chain, for example, in molecular weight or the formation of new chemical groups, arising from radical chain cleavages through mechanochemical processes or through UV radiation in the presence of oxygen. Stabilizers are therefore used to prevent or at least delay said aging. Important representatives of stabilizers are antioxidants, which interfere with the free radicals formed during autoxidation and thus interrupt the degradation process. A distinction is made generally between primary antioxidants, which are able to react directly with oxygen-containing free radicals or C radicals, and secondary antioxidants, which react with hydroperoxides formed as intermediates (see C. Kröhnke et al. Antioxidants in Ullmann's encyclopedia of industrial chemistry, Wiley-VCH Verlag, Weinheim 2015). Typical representatives of primary antioxidants are, for example, phenolic antioxidants, amines, but also lactones. Classes of secondary antioxidants are phosphorus compounds such as phosphites and phosphonites, but also organosulfur compounds such as thioesters and disulfides. Primary and secondary antioxidants are typically frequently combined in practice, which produces a synergistic effect.

Plastics formed from fossil raw materials such as petroleum or natural gas are increasingly being supplemented or replaced by plastics based on renewable raw materials obtained via biochemical processes. The question of sustainability then also arises for the primary and secondary antioxidants used therefor (and for plastics made from fossil raw materials). There is therefore a need for stabilizers based on renewable and available raw materials that are highly effective, have low volatility and are compatible with polymeric substrates.

Basically, primary antioxidants made from renewable raw materials, which are also occasionally used in plastics, are known. Tocopherols (vitamin E) are a typical example. Tocopherols like customary antioxidants have a sterically hindered phenol structure and can be used alone or in combination with secondary antioxidants (e.g. S. Al-Malaika, Macromol. Symp. 2001, 176, 107-117). Tocopherols can e.g. be isolated from natural products such as wheat germ oil or olive oil.

Further known phenols acting antioxidatively in plastics are e.g. quercetin (B. Kirschweng et al., Eur. Pol. J. 2018, 103, 228-237), dihydromyricetin (B. Kirschweng et al., Pol. Degr. Stab. 2016, 133, 192-200), Derivatives of rosmarinic acid (K. Doudin et al., Pol. Degr. Stab. 2016, 130, 126-134) or also tannin (W. J. Grigsby et al., Polymers 2013, 5, 344-360).

Derivatives of ferulic acid are furthermore also known (A. F. Reano et al., ACS Sustainable Chemistry and Engineering 4 (2015), 6562-6571) and caffeic acid (V. Ambrogi et al., Biomacromolecules 15 (2014), 302-310).

Most natural phenols, however, require a high effort in the isolation, the filtration, or the manufacture of applicable daughter products.

Ferulic acid and its salts are, for example, used in the cosmetics industry or as active pharmaceutical ingredients (e.g. FR 2907338, CN 101181256, DE 1957433); the preparation of the salts is generally known (e.g. AT 317184). Layer compounds having light stabilizers that may, by way of example, include sodium cinnamate as possible light stabilizers and that can be used in plastics or coatings are synthesized in CN 107629310.

Ester derivatives of ferulic acid are furthermore also known (A. F. Reano et al., ACS Sustainable Chemistry and Engineering 4 (2015), 6562-6571, A. F. Reano et al., ACS Sustainable Chemistry and Engineering 3 (2015), 3486-3496, oligomers and polymers of ferulic acid (US 2016257846) and caffeic acid (V. Ambrogi et al. Biomacromolecules 15 (2014), 302-310). However, said derivatives are produced by enzymatic syntheses in a relatively complex manner. Ferulic acid derivatives likewise known in the form of ester compounds are isosorbide esters (US2007/0189990) and cholestanyl esters (WO2018/153917).

Starting from this, it was the object of the present invention to provide sustainable antioxidants for organic materials, in particular polymers, on the basis of renewable resources having high efficacy, high thermostability, and small volatility.

This object is achieved with respect to the use of a compound of the general formula I defined in claim 1 for stabilizing organic materials, in particular against oxidative, thermal and/or actinic degradation. This object is furthermore achieved by an organic material in accordance with claim 11. The invention further relates to a method of stabilizing organic materials as specified in claim 14. Specific cinnamic acid salts that are used as stabilizers are defined in claim 15. The present invention furthermore relates to a stabilizer composition in accordance with claim 16. The respective dependent claims in this respect set forth advantageous further developments.

In accordance with a first aspect, the present invention thus relates to the use of a compound or of mixtures of a plurality of compounds in accordance with the general formula I

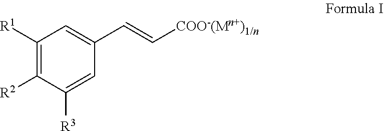

where
R¹, R² and R³ are each selected independently of one another from the group consisting of hydroxy, linear or branched alkoxy groups having 1 to 6 carbon atoms, and hydrogen, with the proviso that at least one of the residues R¹, R² and R³ is a hydroxy residue,
M is selected from the group consisting of metals, and
n is an integer from 1 to 4,
for stabilizing organic materials, in particular against oxidative, thermal and/or actinic degradation.

In accordance with the invention, cosmetics are not counted as belonging to the organic materials.

In accordance with the invention, a metal salt of a hydrocinnamic acid in which at least one phenol group has a steric hindrance is used as the stabilizer.

It has surprisingly been found that the compounds in accordance with the invention in accordance with Formula I act as stabilizers and have a high efficacy, environmental friendliness, and a favorable cost structure on the basis of renewable resources.

A preferred embodiment provides that the invention relates to the stabilization of plastics, coatings, lubricants, hydraulic oils, engine oils, turbine oils, transmission oils, metal machining oils, chemicals, or monomers. For example, plastics in the form of injection molded parts, foils or films, foams, fibers, cables and pipes, sections, hollow bodies, ribbons, membranes, such as geo-membranes, or adhesives that are manufactured by extrusion, injection molding, blow molding, calendering, pressing processes, spinning processes, or rotomolding, e.g. for the electrical and electronic industry, the construction industry, the transport industry (automobile, aircraft, ship, railroad), for medical applications, for domestic and electric appliances, vehicle parts, consumer products, packaging, furniture, textiles. A further area of use includes lacquers, paints, and coatings as well as the stabilization of oils and fats.

A preferred embodiment provides that

the residues R¹, R² und R³ each represent a hydroxy residue,
two of the residues R¹, R² und R³ represent a hydroxy residue and one of the residues R¹, R² und R³ represents hydrogen or a linear or branched alkoxy group having 1 to 6 carbon atoms,
one of the residues R¹, R² und R³ represents a hydroxy residue and two of the residues R¹, R² und R³ represent a linear or branched alkoxy group having 1 to 6 carbon atoms, or one each of the residues R¹, R² and R³ represents a hydroxy residue, a linear or branched alkoxy group having 1 to 6 carbon atoms, and hydrogen.

In accordance with a particularly preferred embodiment, the compound in accordance with general formula I is selected from the group consisting of the following compounds:

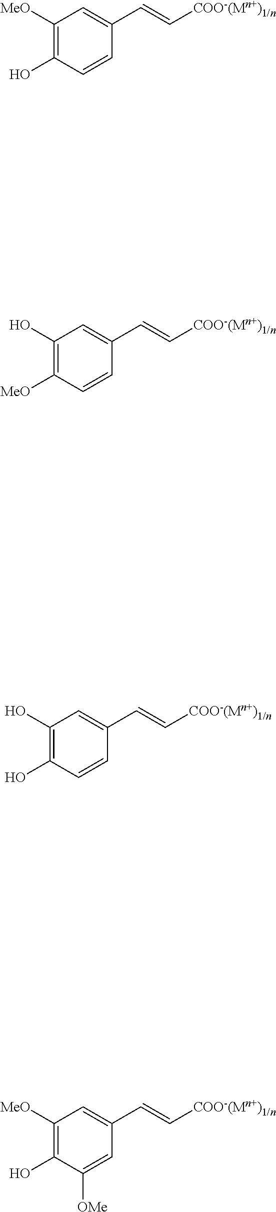

where M and n are defined as above.

In accordance with this preferred embodiment, the hydroxycinnamic acid sales used in accordance with the invention are thus derived from the following hydroxycinnamic acids:

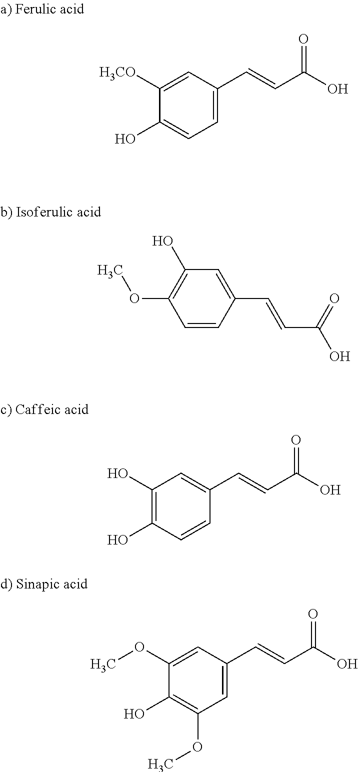 [SMILES: COc1cc(/C=C/C(=O)O)ccc1O ; COc1ccc(/C=C/C(=O)O)cc1O ; O=C(O)/C=C/c1ccc(O)c(O)c1 ; COc1cc(/C=C/C(=O)O)cc(OC)c1O]

The metals M are here in particular selected from the group consisting of alkali metals, alkaline earth metals, aluminum, and zinc.

Preferred alkali metals here are lithium, sodium, and potassium, with sodium being particularly preferred. Preferred alkaline earth metals are in particular magnesium and calcium.

A further preferred embodiment of the present invention provides that the compound in accordance with general formula I or in the case of a mixture of a plurality of compounds in accordance with general formula I, the totality of all the compounds in accordance with general formula I is included in the organic material at a weight proportion of 0.01 to 10.00 wt. %, preferably of 0.02 to 5.00 wt. %, particularly preferably of 0.05 to 2.00 wt,%.

The present invention is in particular suitable for the stabilization of thermoplastic, elastomer, or thermosetting polymers. Thermoplastic and thermosetting polymers are, for example:
a) polymers of olefins or diolefins such as polyethylene (LDPE, LLDPE, VLDPE. ULDPE, MDPE, HDPE, and UHMWPE), metallocene PE (m-PE), polypropylene, polyisobutylene, poly-4-methyl-pentene-1, polybutadiene, polyisoprene, such as also natural rubber (NR), polycyclooctene, polyalkylene carbon monoxide copolymers, and copolymers in the form of statistical or block structures such as polypropylene-polyethylene (EP), EPM or EPDM with, e.g., 5-ethylidene-2-norbornene as a comonomer, ethylene vinyl acetate (EVA), ethyleneacrylic ester such as ethylene butyl acrylate, ethylene-acrylic acid and their salts (ionomers), and terpolymers such as ethylene acrylic acid glycidyl(meth)acrylate, graft polymers such as polypropylene g-maleic acid anhydride, polypropylene graft-acrylic acid, polyethylene graft acrylic acid, polyethylene polybutylacrylate graft maleic acid hydride, and blends such as LDPE/LLDPE or also long chain branched polypropylene copolymers that are prepared with alpha olefins as comonomers such as with 1-butene, 1-hexene, 1-octene, or 1-octadecene.
b) polystyrene, polymethyl styrene, poly-alpha-methyl styrene, polyvinyl naphthalene, polyvinyl biphenyl, polyvinyl toluol, styrene butadiene (SB), styrene butadiene styrene (SBS), styrene ethylene butylene styrene (SEBS), styrene ethylene propylene styrene, styrene isoprene, styrene isoprene styrene (SIS), styrene butadiene acrylonitrile (ABS), styrene acrylonitrile (SAN), styrene acrylonitrile acrylate (ASA), styrene ethylene, styrene maleic acid anhydride polymers, incl. corresponding graft copolymers such as styrene on butadiene, maleic acid anhydride on SBS or SEBS, and graft copolymers of methylmethacrylate, styrene butadiene, and ABS (MABS), and hydrated polystyrene derivatives such as polyvinyl cyclohexane
c) halogen-containing polymers such as polyvinyl chloride (PVC), polychloroprene and polyvinylidene chloride (PVDC), copolymers of vinyl chloride and vinylidene chloride or of vinyl chloride and vinyl acetate, chlorinated polyethylene, polyvinylidene fluoride, epichlorohydrin homo and copolymers, in particular with ethylene oxide (ECO)
d) polymers of unsaturated esters such as polyacrylates and polymethacrylates such as polymethyl methacrylate (PMMA), polybutyl acrylate, polyauryl acrylate, poly stearyl acrylate, polyglycidyl methacrylate, polyacrylonitrile, polyacrylamides, copolymers such as polyacrylonitrile-poly alkyl acrylate,
e) polymers of unsaturated alcohols and derivatives, such as e.g. polyvinyl alcohol, polyvinyl acetate, polyvinyl butyral, polyallyl phthalate, polyallyl melamine
f) polyacetals, such as e.g. polyoxymethylene (POM) or copolymers with, for example, butanal,
g) polyphenylene oxides and blends with polystyrene or polyamides,
h) polymers of cyclic ethers such as polyethylene glycol, polypropylene glycol, polyethylene oxide, polypropylene oxide, polytetrahydrofuran,
i) polyurethanes, made from hydroxy-terminated polyethers or polyesters and aromatic or aliphatic isocyanates such as 2,4- or 2,6-tolylene diisocyanate or methylenediphenyl diisocyanate, in particular linear polyurethanes (TPU), polyureas,
j) polyamides such as polyamide-6, 6.6, 6.10, 4.6, 4.10, 6.12, 10.10, 10.12, 12.12, polyamide 11, polyamide 12 and (partially) aromatic polyamides such as polyphthalamides, for example, made from terephthalic acid and/or isophthalic acid and aliphatic diamines such as hexamethylenediamine or m-xylylenediamine or from aliphatic dicarboxylic acids such as adipic acid or sebacic acid and aromatic diamines such as 1,4- or 1,3-diaminobenzene, blends of different polyamides such as PA-6 and PA 6.6 or blends of polyamides and polyolefins such as PA/PP
k) polyimides, polyamide-imides, polyether imides, polyester imides, poly(ether)ketones, polysulfones, polyether sulfones, polyaryl sulfones, polyphenylene sulfides, polybenzimidazoles, polyhydantoins,
l) polyesters made from aliphatic or aromatic dicarboxylic acids and diols or from hydroxycarboxylic acids such as polyethylene terephthalate (PET), polybutylene terephthalate (PBT), polypropylene terephthalate (PTT), polyethylene naphthylate (PEN), poly-1,4-dimethylolcyclohexane terephthalate, polyhydroxybenzoate, polyhydroxynaphthalate, polyhydroxy acid (PLA), polyhydroxybutyrate (PHB), polyhydroxyvalerate (PHV), polyethylene succinate, polytetramethylene succinate, polycaprolactone
m) polycarbonates, polyester carbonates and blends such as PC/ABS, PC/PBT, PC/PET/PBT, PC/PA
n) cellulose derivatives, such as e.g. cellulose nitrate, cellulose acetate, cellulose propionate, cellulose butyrate,
o) epoxy resins consisting of di- or polyfunctional epoxy compounds in combination with, for example, hardeners based on amines, anhydrides, dicyandiamide, mercaptans, isocyanates or catalytic hardeners,
p) phenol resins such as phenol formaldehyde resins, urea formaldehyde resins, melamine formaldehyde resins,
q) unsaturated polyester resins of unsaturated dicarboxylic acids and diols with vinyl compounds such as styrene, alkyd resins, allyl resins,
r) silicones, e.g. based on dimethyl siloxanes, methyl phenyl siloxanes, or diphenyl siloxanes, e.g. vinyl group terminated
s) and mixtures, combinations or blends of two or more of the aforementioned polymers.

If the polymers specified under a) to r) are copolymers, these can exist in the form of statistical (“random”), block or “tapered” structures. Furthermore, the polymers mentioned can exist in the form of linear, branched, star-shaped or hyperbranched structures.

If the polymers specified under a) to r) are stereoregular polymers, they can exist in the form of isotactic, stereotactic, but also atactic forms or as stereoblock copolymers.

Furthermore, the polymers specified under a) to r) can have both amorphous and (partially) crystalline morphologies.

Optionally, the polyolefins mentioned under a) can also be crosslinked, for example crosslinked polyethylene, which is then referred to as X-PE.

Furthermore, the present compounds can be used to stabilize rubbers and elastomers. This can be natural rubber (NR) or synthetic rubber materials such as NR (Natural Rubber), chloroprene (CR), polybutadiene (BR), styrene-butadiene (SBR), polyisoprene (IR), butyl rubber (IIR), nitrile rubber (NBR), hydrogenated nitrile rubber (HNBR), polyester or polyether urethane rubber, silicone rubber.

Apart from new goods, the plastics can be recycled plastics, for example, from industrial collections such as e.g. production waste or plastics from household or recyclable collections.

Thermoplastic plastics, and in particular plastics that are used in packaging such as e.g. food packaging, in particular polyolefins, polystyrene, polyesters and polyamides, are preferred as plastics. Polypropylene homo- and copolymers are very particularly preferred, and polyethylene in the form of LDPE, LLDPE, HDPE, MDPE, VLDPE and polyethylene terephthalate (PET), homo- and copolymers.

Aliphatic polyesters from renewable resources are furthermore in particular preferred that are substantially prepared from aliphatic dicarboxylic acids and aliphatic diols, from hydroxy carboxylic acids or lactones such as polylactic acid (PLA), polyglycolic acid (PGA), polyhydroxy butyric acid (PHB), polyhydroxy valeric acid (PHV), polyethylene succinate (PESu), polybutylene succinate (PBS), polyethylene adipate polybutylene succinate coadipate (PBSA), or polycaprolactone (PCL).

In accordance with the invention, the plastics can in particular be present in the form of injection molded parts, foils or films, foams, fibers, cables and pipes, sections, hollow bodies, ribbons, membranes, e.g. geo-membranes, or adhesives that are manufactured by extrusion, injection molding, blow molding, calendering, pressing processes, spinning processes, rotomolding, e.g. for the electrical and electronic industry, the construction industry, the transport industry (automobile, aircraft, ship, railroad), for medical applications, for domestic and electric appliances, vehicle parts, consumer products, packaging, furniture, textiles. A further area of use includes lacquers, paints, and coatings as well as the stabilization of oils and fats.

A further advantageous embodiment of the use in accordance with the invention is characterized in that the plastic comprises at least one further additive, selected from the group consisting of primary and/or secondary antioxidants, in particular primary and/or secondary antioxidants selected from the group consisting of phosphites, phosphonites, thiols, phenolic antioxidants, sterically hindered amines, hydroxylamines, and mixtures or combinations thereof, UV absorbers, light stabilizers, stabilizers on a hydroxyl amine base, stabilizers on a benzofuranone base, nucleating agents, toughening agents, plasticizers, mold lubricants, rheological modifiers, chain extenders, processing aids, pigments, dyes, optical brighteners, antimicrobial active agents, antistatic agents, slip agents, anti-blocking agents, coupling agents, dispersing agents, compatibilizers, oxygen scavengers, acid scavengers, costabilizers, marking agents, and anti-fogging agents, and/or is added to the plastic on use.

Suitable primary antioxidants (A) are phenolic antioxidants, amines, and lactones

Suitable synthetic phenolic antioxidants are, for example: Alkylated monophenols, such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, linear or branched nonylphenols such as 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof;

alkylthiomethyl phenols, such as 2,4-dioctylthiomethyl-6-tert-butylphenol, 2,4-dioctylthiomethyl-6-methylphenol, 2,4-dioctylthiomethyl-6-ethylphenol, 2,6-didodecylthiomethyl-4-nonylphenol;
hydroquinones and alkylated hydroquinones, such as e.g. 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxylphenyl)adipate;
tocopherols, such as α-, β-, γ-, δ-tocopherol and mixtures hereof (vitamin E); hydroxylated thiodiphenyl ethers, such as 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide;
alkylide bisphenols such as 2,2′methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclhexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol, 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethyleneglycol-bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane;
O-, N- and S-benzyl compounds such as 3,5,3′,5′-tetra-tert-butyl-4,4′-dihydroxydibenzylether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate;
hydroxybenzylated malonates such as dioctadecyl-2,2-bis(3,5-di-tert-butyl-2-hydroxybenzyl)malonate, dioctadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate;
aromatic hydroxybenzyl compounds, such as 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl) phenol;
triazine compounds, such as 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxphenylethyl)-1,3,5-triazine, 1,3,5-tris(3,5-di-tert-butyl-4-hydroxyphenylpropionyl)hexahydro-1,3,5-triazine, 1,3,5-tris(3,5-dicyclohexyl-4-hydroxybenzyl)isocyanurate;
benzyl phosphonates, such as dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethylester of 3,5-di-tert-butyl-4-hydroxybenzylphosphonic acid;
acylaminophenols, such as 4-hydroxylauranilide, 4-hydroxystearanilide, octyl-N-(3,5-di-tert-butyl-4-hydroxyphenyl)carbamate;
esters of β-(3,5-di-tert-butyl-4-hydroxyphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of β-(5-tert-butyl-4-hydroxy-3-methylphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, 3,9-bis[2-{3-(3-tert-butyl-4-hydroxy-5-methylphenyl)propionyloxy}-1,1-dimethylethyl]-2,4,8,10-tetraoxaspiro[5.5]undecane;
esters of β-(3,5-dicyclohexyl-4-hydroxyphenyl)propionic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of (3,5-di-tert-butyl-4-hydroxyphenyl)acetic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
amides of β-(3,5-di-tert-butyl-4-hydroxyphenyl)propionic acid, such as N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, marketed by Uniroyal); ascorbic acid (vitamin C).

Particularly preferred phenolic antioxidants are the following structures:

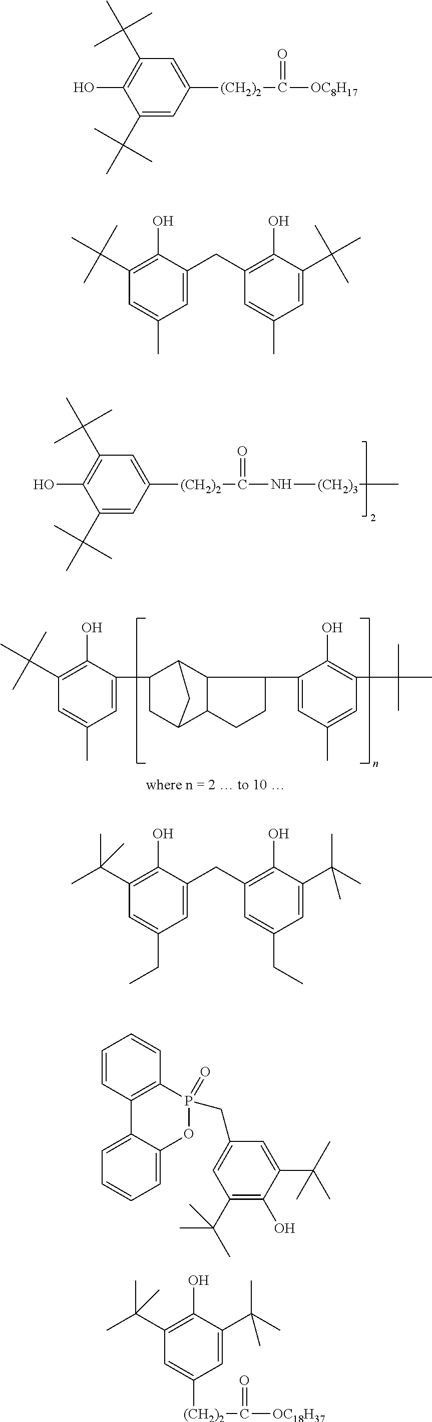

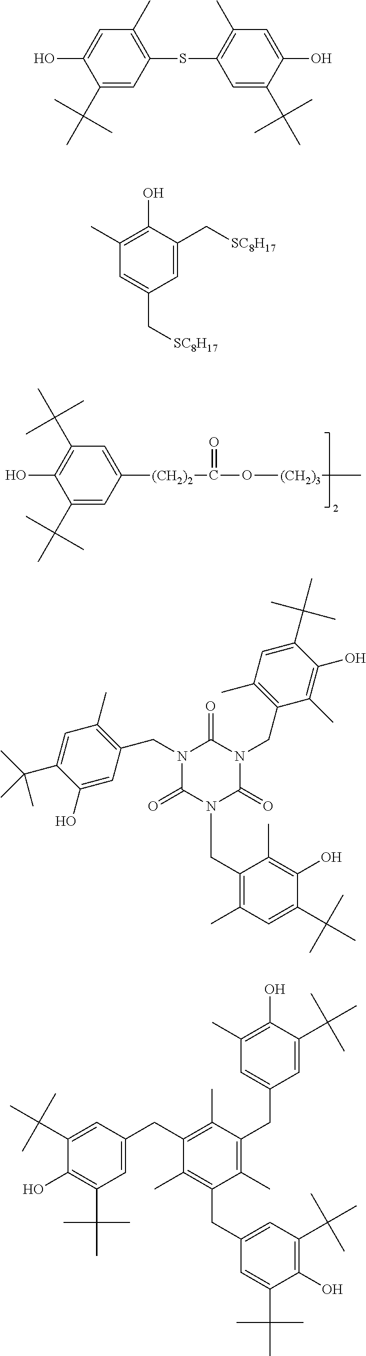

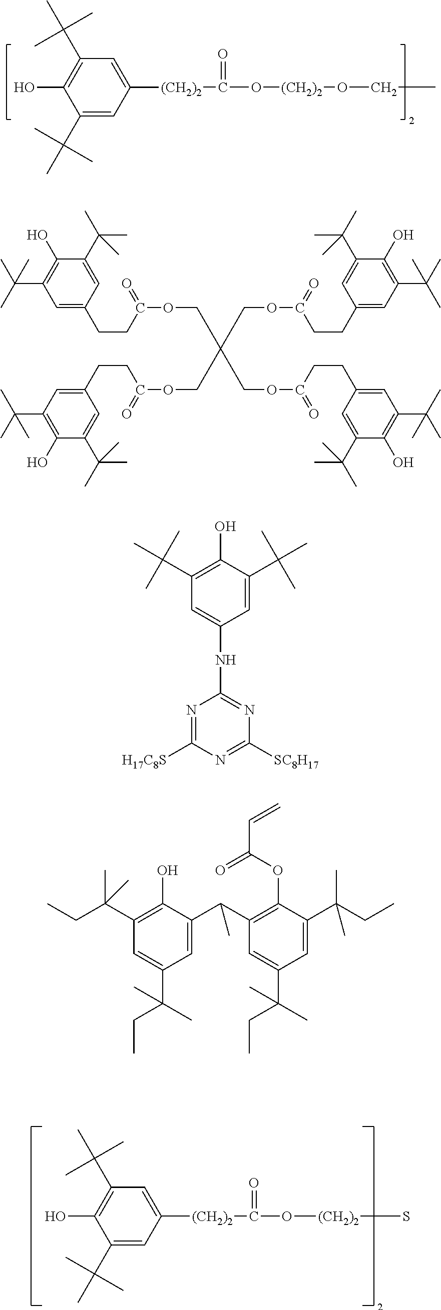

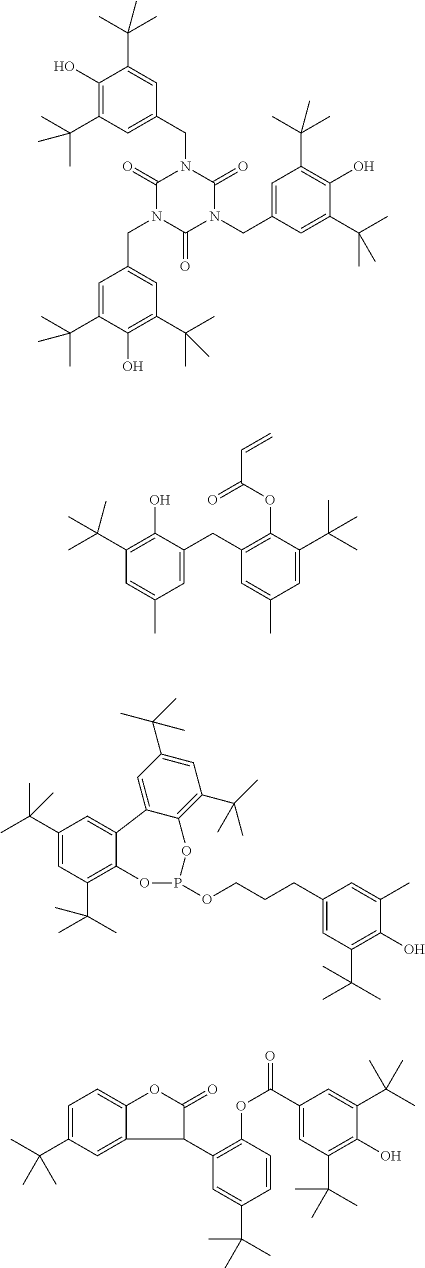

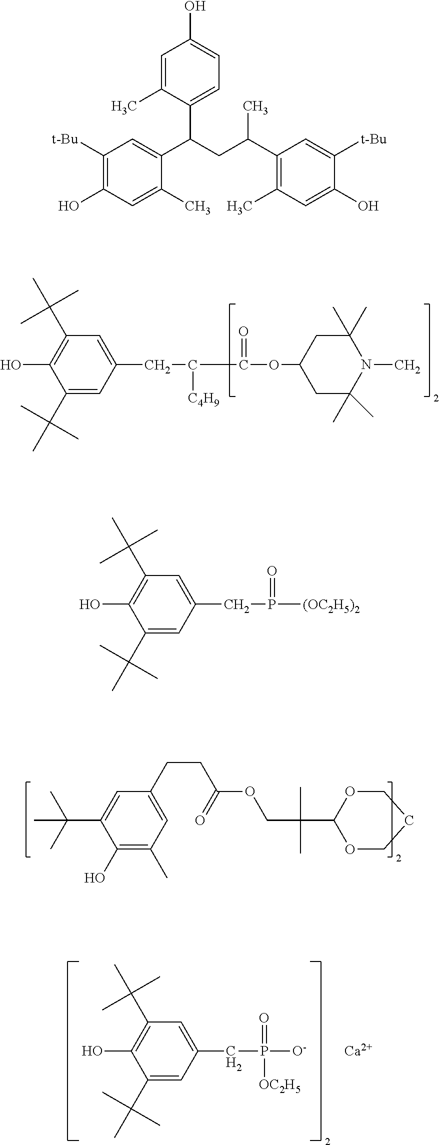

Very particularly preferred phenolic antioxidants are octadecyl-3-(3,5-di-tert-butyl-4-hydroxyphenyl)propionate and pentaerythritol tetrakis(3-(3,5-di-tert-butyl-4-hydroxyphenyl)propionate

Further particularly preferred phenolic antioxidants are based on sustainable raw materials such as tocopherols (vitamin E), tocotrienols, tocomonoenols, carotenoids, hydroxytyrosol, flavonols such as chrysin, quercetin, hesperidin, nehesperidin, naringin, marin, camphor oil, fisetin, anthocyanins such as delphinidin and malvidin, curcumin, carnosic acid, carnosol, rosemarinic acid, tannin, and resveratrol.

Suitable aminic antioxidants are, for example:

N,N′-di-isopropyl-p-phenylene diamine, N,N′-di-sec-butyl-p-phenylene diamine, N,N′-bis(1,4-dimethylpentyl)-p-phenylene diamine, N,N′-bis(1-ethyl-3-methylpentyl)-p-phenylene diamine, N,N′-bis(1-methylheptyl)-p-phenylene diamine, N,N′-dicyclohexyl-p-phenylene diamine, N,N′-diphenyl-p-phenylene diamine, N,N′-bis(2-naphthyl)-p-phenylene diamine, N-isopropyl-N′-phenyl-p-phenylene diamine, N-(1,3-dimethylbutyl)-N′-phenyl-p-phenylene diamine, N-(1-methylheptyl)-N′-phenyl-p-phenylene diamine, N-cyclohexyl-N′-phenyl-p-phenylene diamine, 4-(p-toluene sulfamoyl)diphenylamine, N,N′-dimethyl-N,N′-di-sec-butyl-p-phenylene diamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylamino-phenol, bis(4-methoxyphenyl)amine, 2,6-di-tert-butyl-4-dimethylaminomethyl-phenol, 2,4′-diaminodiphenylmethane, 4,4′-diaminodiphenylmethane, N,N,N′,N′-tetra-methyl-4,4′-diaminodiphenylmethane, 1,2-bis[(2-methyl-phenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazinene, N-allylphenothiazine, N,N,N′,N′-tetraphenyl-1,4-diaminobut-2-ene and mixtures or combinations hereof.

Preferred aminic antioxidants are: N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine

Particularly preferred phenolic antioxidants are the structures:

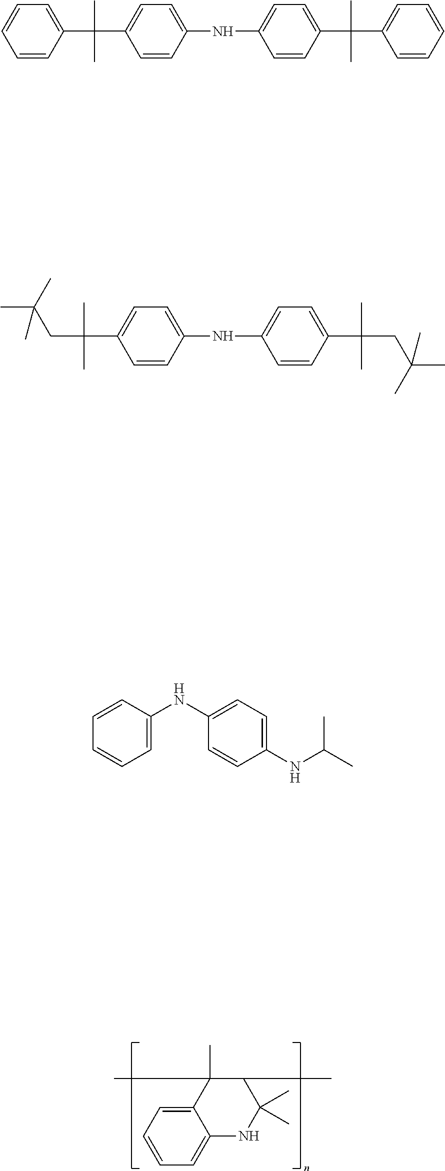

where n=3 to 100.

Further preferred aminic antioxidants are hydroxylamines or N-oxides (nitrones) such as N,N-dialkylhydroxylamines, N,N-dibenzylhydroxylamine, N,N-dilaurylhydroxylamine, N,N-distearylhydroxylamine, N-benzyl-α-phenylnitrone, N-octadecyl-α-hexadecylnitrone, and Genox EP (SI group) in accordance with the formula:

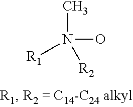

Suitable lactones are benzofuranones and indolinones such as 3-(4-(2-acetoxyethoxy)phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-(2-hydroxyethoxy]phenyl)benzofuran-2-one), 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-dimethylphenyl)-5,7-di-tert-butyl-benzofuran-2-one as well as lactones that additionally contain a phosphite group such as

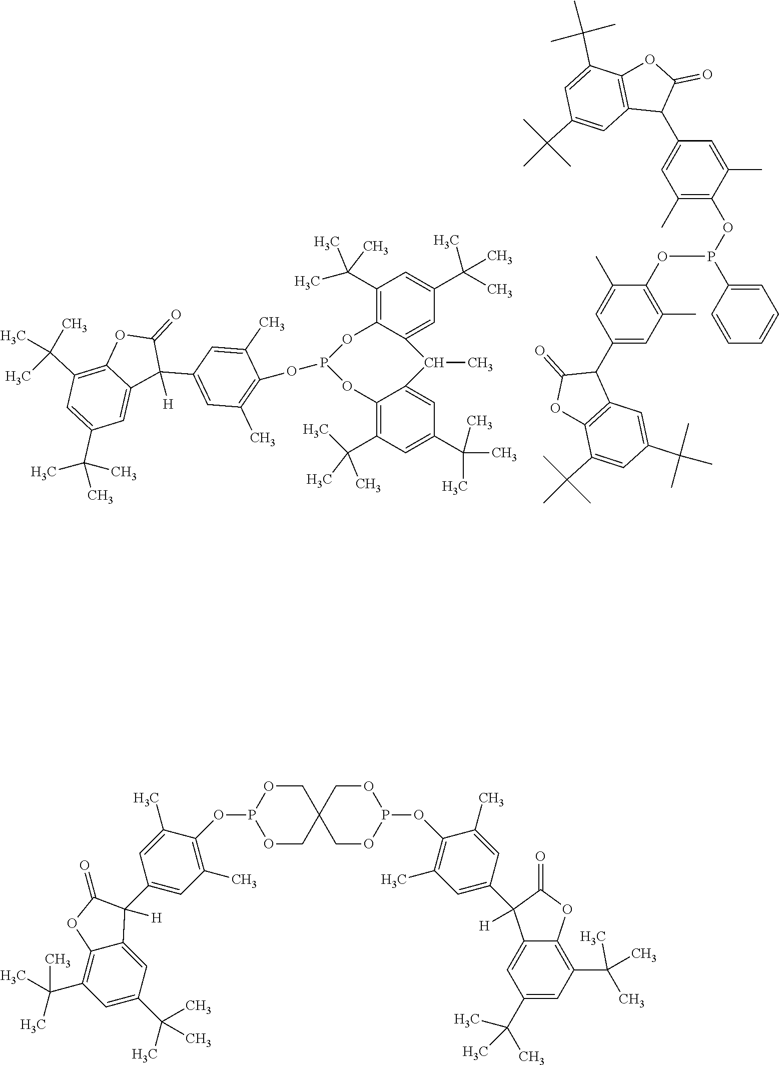

A further suitable group of antioxidants are isoindolol[2,1-A]chinazoniles such as

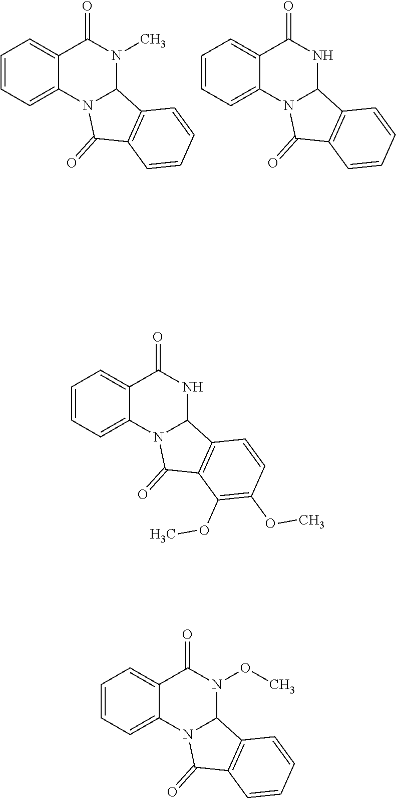

Suitable secondary antioxidants are in particular phosphites or phosphonites such as

triphenylphosphite, diphenylalkylphosphites, phenyldialkylphosphites, tri(nonylphenyl)phosphite, trilaurylphosphites, trioctadecylphosphite, distearylpentaerythritoldiphosphite, tris-(2,4-di-tert-butylphenyl)phosphite, diisodecylpentaerythritoldiphosphite, bis(2,4-di-tert-butylphenyl)pentaerythritoldiphosphite, bis(2,4-di-cumylphenyl)pentaerythritoldiphosphite, bis(2,6-di-tert-butyl-4-methylphenyl)pentaerythritoldiphosphite, diisodecyloxypentaerythritoldiphosphite, bis(2,4-di-tert-butyl-6-methylphenyl)pentaerythritoldiphosphite, bis(2,4,6-tris(tert-butylphenyl)pentaerythritoldiphosphite, tristearylsorbitoltriphosphite, tetrakis(2,4-di-tert-butylphenyl)-4,4′-biphenylene diphosphonite, 6-isooctyloxy-2,4,8,10-tetra-tert-butyl-12H-dibenz[d,g]-1,3,2-dioxaphosphocine, bis(2,4-di-tert-butyl-6-methylphenyl)methylphosphite, bis(2,4-di-tert-butyl-6-methylphenyl)ethylphosphite, 6-fluoro-2,4,8,10-tetra-tert-butyl-12-methyl-dibenz[d,g]-1,3,2-dioxaphosphocine, 2,2′2″-nitrilo[triethyltris(3,3″,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl))phosphite, 5-butyl-5-ethyl-2-(2,4,6-tri-tert-butylphenoxy)-1,3,2-dioxaphosphirane.

Particularly preferred phosphites/phosphonites are:

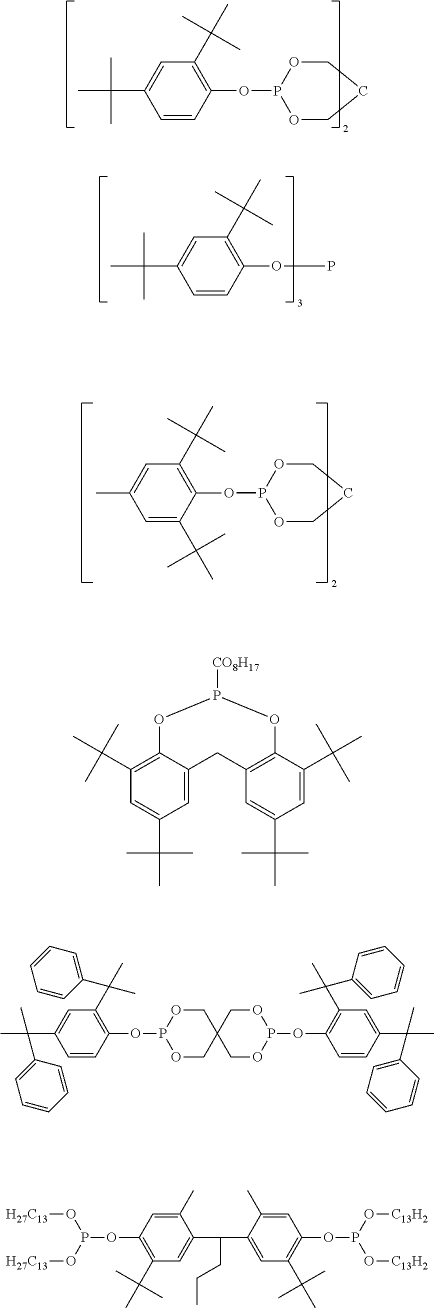

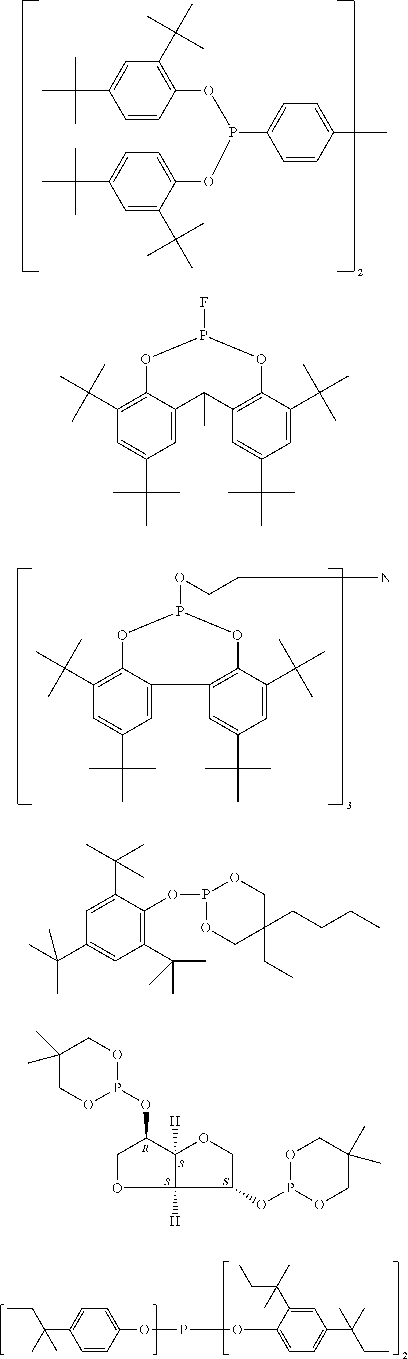

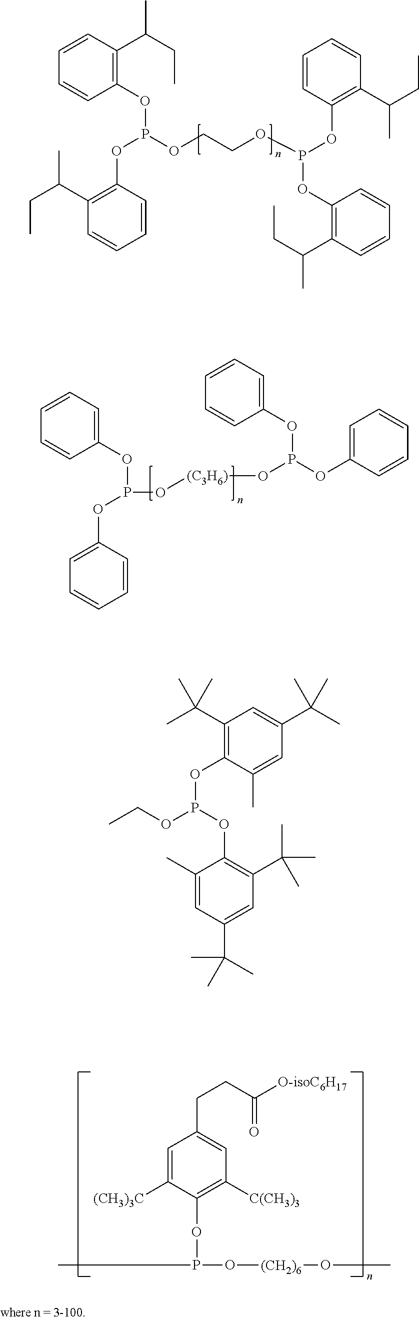

A preferred phosphonite is:

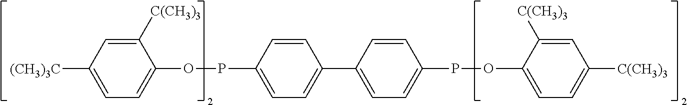

Very particularly preferably, the phosphite tris-(2,4-d-tert-butylphenyl)phosphite is used as the secondary antioxidant.

Suitable secondary antioxidants are furthermore organosulfur compounds such as sulfides and disulfides, e.g. distearylthiodipropionate, dilaurylthiodipropionate; ditridecyldithiopropionate, ditetradecylthiodipropionate, 3-(dodecylthio)-1,1′-[2,2-bis[[3-(dodecylthio)-1-oxopropoxy]methyl]-1,3-propandiyl] propanoic acid ester. The following structures are preferred:

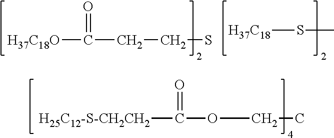

Further suitable organosulfur compounds are sulfurous amino acids, in particular cysteine, cystine, or methionine.

Suitable acid scavengers (“antacids”) are salts of monovalent, bivalent, trivalent, or quadrivalent metals, preferably alkali metals, alkaline earth metals, aluminum or zinc, in particular formed with fatty acids such as calcium stearate, magnesium stearate, zinc stearate, aluminum stearate, calcium laurate, calcium behenate, calcium lactate, calcium stearoyl-2-lactate. Further classes of suitable acid scavengers are hydrotalcites, in particular synthetic hydrotalcites on the basis of aluminum, magnesium and zinc, hydrocalumites, zeolites, alkaline earth metals, in particular calcium oxide and magnesium oxide and zinc oxide, alkaline earth carbonates, in particular calcium carbonate, magnesium carbonate and dolomite, and hydroxides, in particular brucite (magnesium hydroxide).

Suitable costabilizers are furthermore polyols, in particular alditols or cyclitols. Polyols are e.g. pentaerythritol, dipentaerythritol, tripentaerythritol, short chain polyether polyols or short chain polyester polyols, and hyperbranched polymers/oligomers, or dendrimers having alcohol groups e.g.

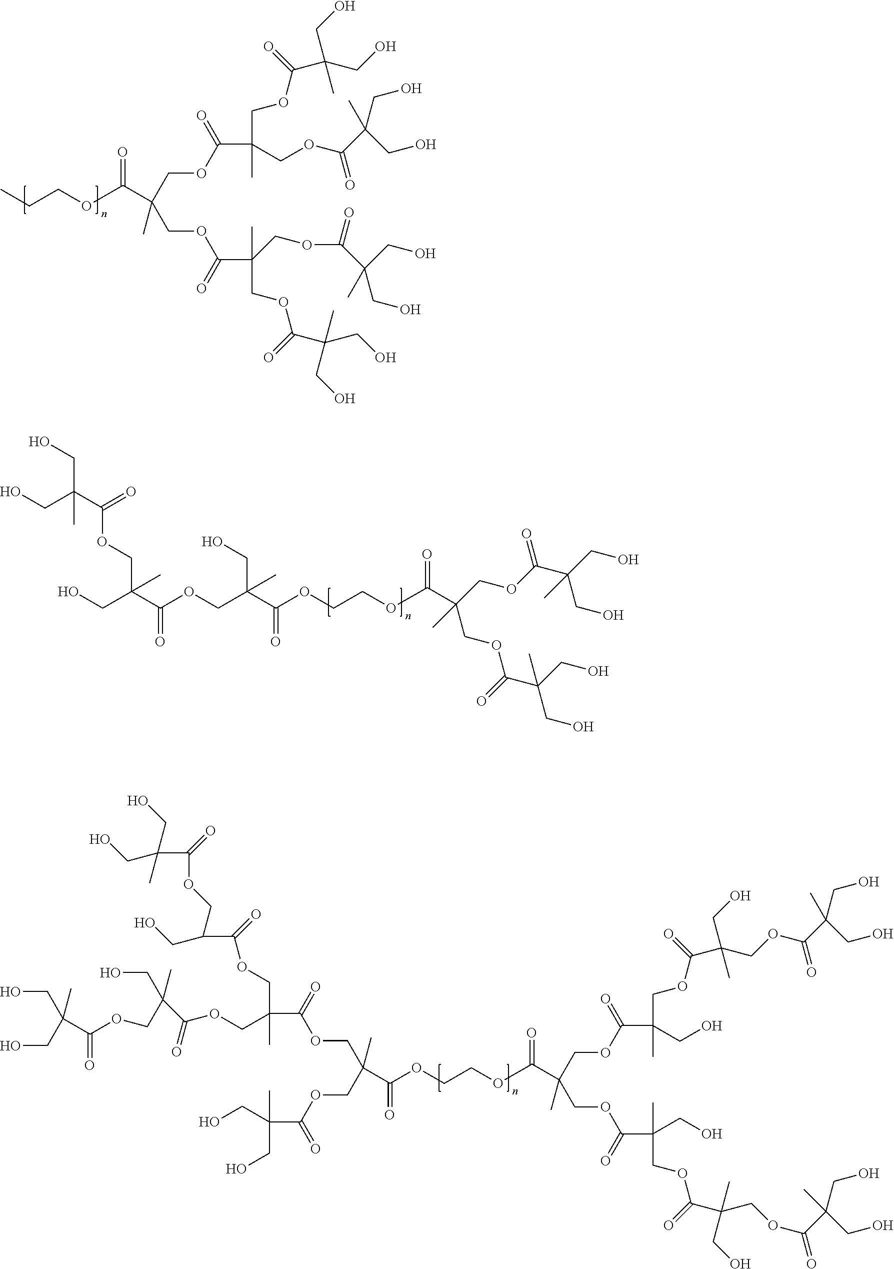

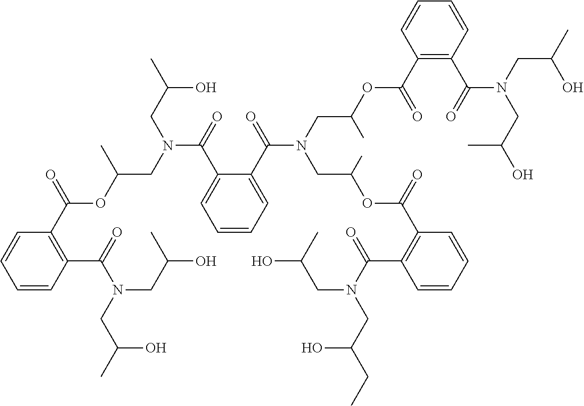 [SMILES: CCC(O)CN(CC(C)O)C(=O)c1ccccc1C(=O)OC(C)CN(CC(C)OC(=O)c1ccccc1C(=O)N(CC(C)O)CC(C)O)C(=O)c1ccccc1C(=O)N(CC(C)O)CC(C)OC(=O)c1ccccc1C(=O)N(CC(C)O)CC(C)O]

The at least one alditol is preferably selected from the group consisting of threitol, erythritol, galactitol, mannitol, ribitol, sorbitol, xylitol, arabitol, isomalt, lactitol, maltitol, altritol, iditol, maltotritol and hydrogenated oligo- and polysaccharides having polyol end groups and mixtures thereof. The at least one preferred alditol is particularly preferably selected from the group comprising erythritol, mannitol, isomaltol, maltitol, and mixtures thereof.

Examples for further suitable sugar alcohols are heptitols and octitols: meso-glycero-allo-heptitol, D-glycero-D-altro-heptitol, D-glycero-D-manno-heptitol, meso-glycero-gulo-heptitol, D-glycero-D-galacto-heptitol (perseitol), D-glycero-D-gluco-heptitol, L-glycero-D-gluco heptitol, D-erythro-L-galacto-octitol, D-threo-L-galacto-octitol.

The at least one cyclitol can in particular be selected from the group consisting of inositol (myo, scyllo-, D-chiro-, L-chiro-, muco-, neo-, allo-, epi-und cis-inositol), 1,2,3,4-tetrahydroxycyclohexane, 1,2,3,4,5-pentahydroxycyclohexane, quercitol, viscumitol, bornesitol, conduritol, ononitol, pinitol, pinpollitol, quebrachitol, ciceritol, quinic acid, shikimic acid, and valienol, with myo-inositol (myo-inositol) being preferred here.

Suitable light stabilizers are, for example, compounds based on 2-(2′-hydroxyphenyl) benzotriazoles, 2-hydroxy benzophenones, esters of benzoic acids, acrylates, oxamides, and 2-(2-hydroxyphenyl)-1,3,-5-triazines.

Suitable 2-(2′-hydroxyphenyl)benzotriazoles are, for example, 2-(2′-hydroxy-5′methylphenyl)benzotriazole, 2-(3′,5′-di-tert-butyl-2′-hydroxyphenyl)benzotriazole, 2-(5′-tert-butyl-2′-hydroxy-phenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorbenzotriazole, 2-(3′-tert-butyl-2′-hydroxy-5′-methylphenyl-5-chlorbenzotriazole, 2-(3′-sec-butyl-5′-tert-butyl-2′-hydroxy-phenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(α,α-dimethylbenzyl)-2′-hydroxyphenyl)benzotriazole, 2-(3′-tert-butyl-2′-hydroxy-5′-(2-octyloxycarbonylethyl)phenyl)-5-chlorbenzotriazole, 2-(3′-tert-butyl-5′-[2-(2-ethylhexyloxy)carbonylethyl]-2′-hydroxyphenyl)-5-chlorbenzotriazole, 2-(3′-tert-butyl-2′-hydroxy-5′-(2-methoxycarbonylethyl)phenyl)-5-chlorbenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazol-2-ylphenol]; the product of the transesterification of 2-[3′-tert-butyl-5′-(2-methoxycarbonylethyl)-2′-hydroxyphenyl]-2H-benzotriazole with polyethyleneglycol 300; [R—CH₂CH₂—COO—CH₂CH₂]₂, where R=3′-tert-butyl-4′-hydroxy-5′-2H-benzotriazole-2-ylphenyl, 2-[2′-hydroxy-3′-(α,α-dimethylbenzyl)-5′-(1,1,3,3-tetramethylbutyl)phenyl]benzotriazole, 2-[2′-hydroxy-3′-(1,1,3,3-tetramethylbutyl)-5′-(α,α-dimethylbenzyl)phenyl]benzotriazole.

Suitable 2-hydroxybenzophenones are, for example, 4-hydroxy-, 4-methoxy-, 4-octyloxy-, 4-decyloxy-4-dodecyloxy, 4-benzyloxy, 4,2′,4′-trihydroxy- and 2′-hydroxy-4,4′-dimethyoxy derivatives of the 2-hydroxy benzophenones.

Suitable acrylates are, for example, ethyl-α-cyano-β,β-diphenylacrylate, isooctyl-α-cyano-β,β-diphenylacrylate, methyl-α-carbomethoxycinnamate, methyl-α-cyano-β-methyl-β-methoxycinnamate, butyl-α-cyano-β-methyl-β-methoxycinnamate, methyl-α-carbomethoxy-β-methoxycinnamate and N-(β-carbomethoxy-β-cyanovinyl)-2-methylindoline.

Suitable esters of benzoic acids are, for example, 4-tert-butylphenylsalicylate, phenylsalicylate, octylphenylsalicylate, dibenzoylresorcinol, bis(4-tert-butylbenzoyl)resorcinol, benzoylresorcinol, 2,4-di-tert-butylphenyl-3,5-di-tert-butyl-4-hydroxybenzoate, hexadecyl-3,5-di-tert-butyl-4-hydroxybenzoate, octadecyl-3,5-di-tert-butyl-4-hydroxybenzoate, 2-methyl-4,6-di-tert-butylphenyl-3,5-di-tert-butyl-4-hydroxybenzoate.

Suitable oxamides are, for example, 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixtures with 2-ethoxy-2′-ethyl-5,4′-di-tert-butoxanilide, mixtures of o- and p-methoxy-disubstituted oxanilides and mixtures of o- and p-ethoxy-disubstituted oxanilides.

Suitable 2-(2-hydroxyphenyl)-1,3,5-triazines are, for example, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)-phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl-1,3,5-triazine.

Suitable metal deactivators are, for example, N,N′-diphenyloxamide, N-salicylal-N′-salicyloylhydrazine, N,N′-bis(salicyloyl)hydrazine, N,N′-bis(3,5-di-tert-butyl-4-hydroxyphenylpropionyl)hydrazin, 3-salicyloylamino-1,2,4-triazole, bis(benzylidene)oxalyldihydrazide, oxanilide, isophthaloyldihydrazide, sebacoyl-bis-phenylhydrazide, N,N′-diacetyladipoyldihydrazide, N,N′-bis(salicyloyl)oxylyldihydrazide, N,N′-bis(salicyloyl)thiopropionyldihydrazide.

Particularly preferred as metal deactivators are

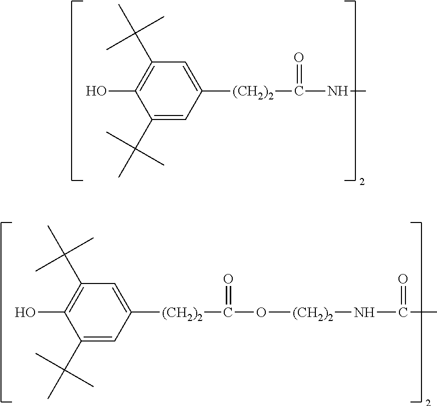

Suitable hindered amines are, for example 1,1-bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl)-n-butyl-3,5-di-tert-butyl-4-hydroxybenzylmalonaet, the condensation product from 1-(2-hydroxyethyl)-2,2,6,6-tetramethyl-4-hydroxypiperidine and succinic acid, linear or cyclic condensation products of N,N′-bis(2,2,6,6-tetramethyl-4-piperidyl)hexamethylene diamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butane tetracarboxylate, 1,1′-(1,2-ethandiyl)-bis(3,3,5,5-tetramethylpiperazinone), 4-benzoyl-2,2,6,6-tetramethylpiperidine, 4-stearyloxy-2,2,6,6-tetramethylpiperidine, linear or cyclic condensation products of N,N′-bis(2,2,6,6-tetramethyl-4-piperidyl)hexamethylene diamine and 4-morpholino-2,6-dichloro-1,3,5-triazine, the reaction product of 7,7,9,9-tetramethyl-2-cycloundecyl-1-oxa-3,8-diaza-4-oxospiro-[4,5]decane and epichlorohydrine.

The sterically hindered N—H, N-alkyl such as N-methyl or N-octyl, the N-alkoxy derivatives such as N-methoxy or N-octyloxy, the cycloalkyl derivatives such as N-cyclohexyloxy and the N-(2-hydroxy-2-methylpropoxy) analogs are also each included in the above-given structures here.

Preferred hindered amines furthermore have the following structures:

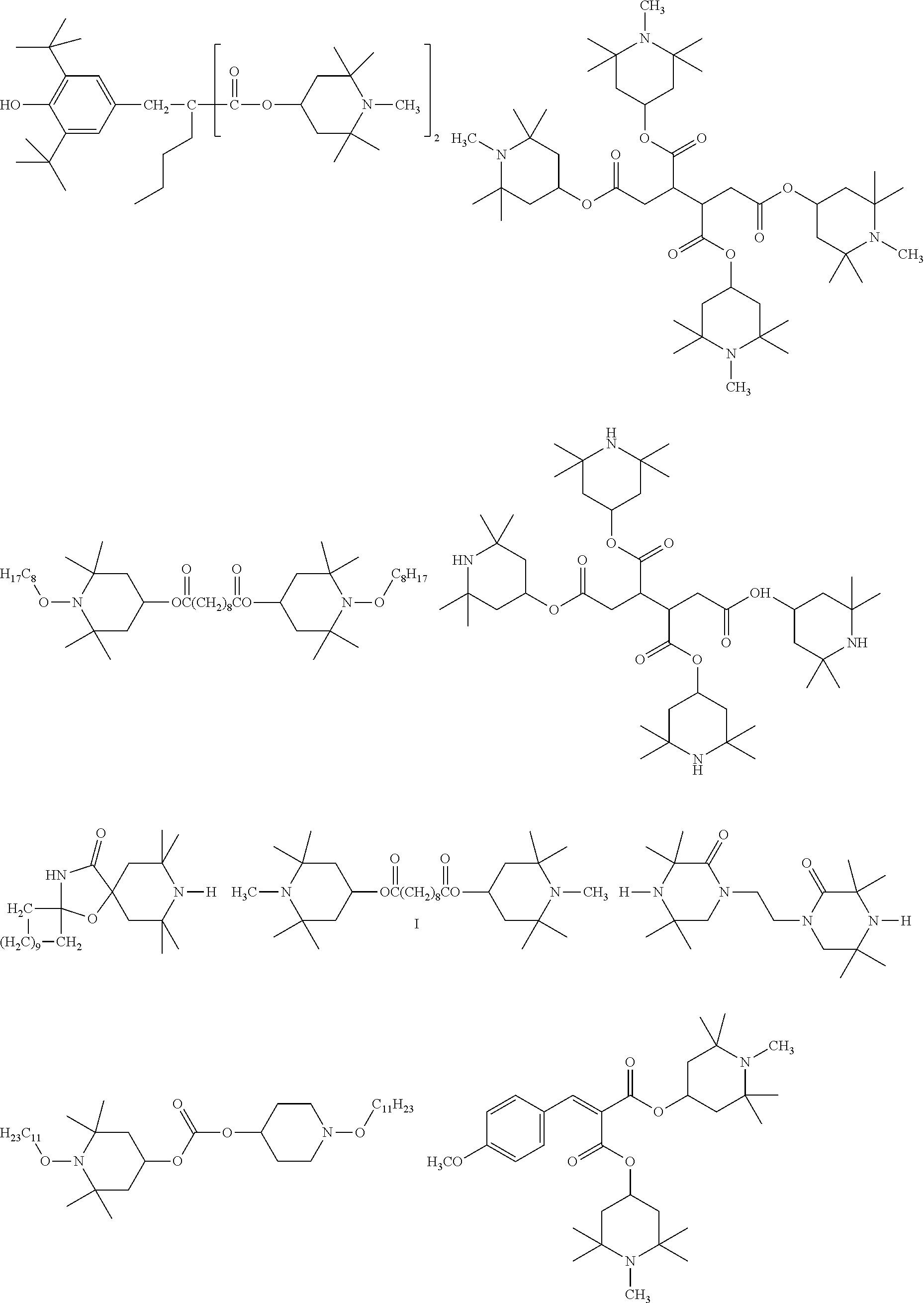

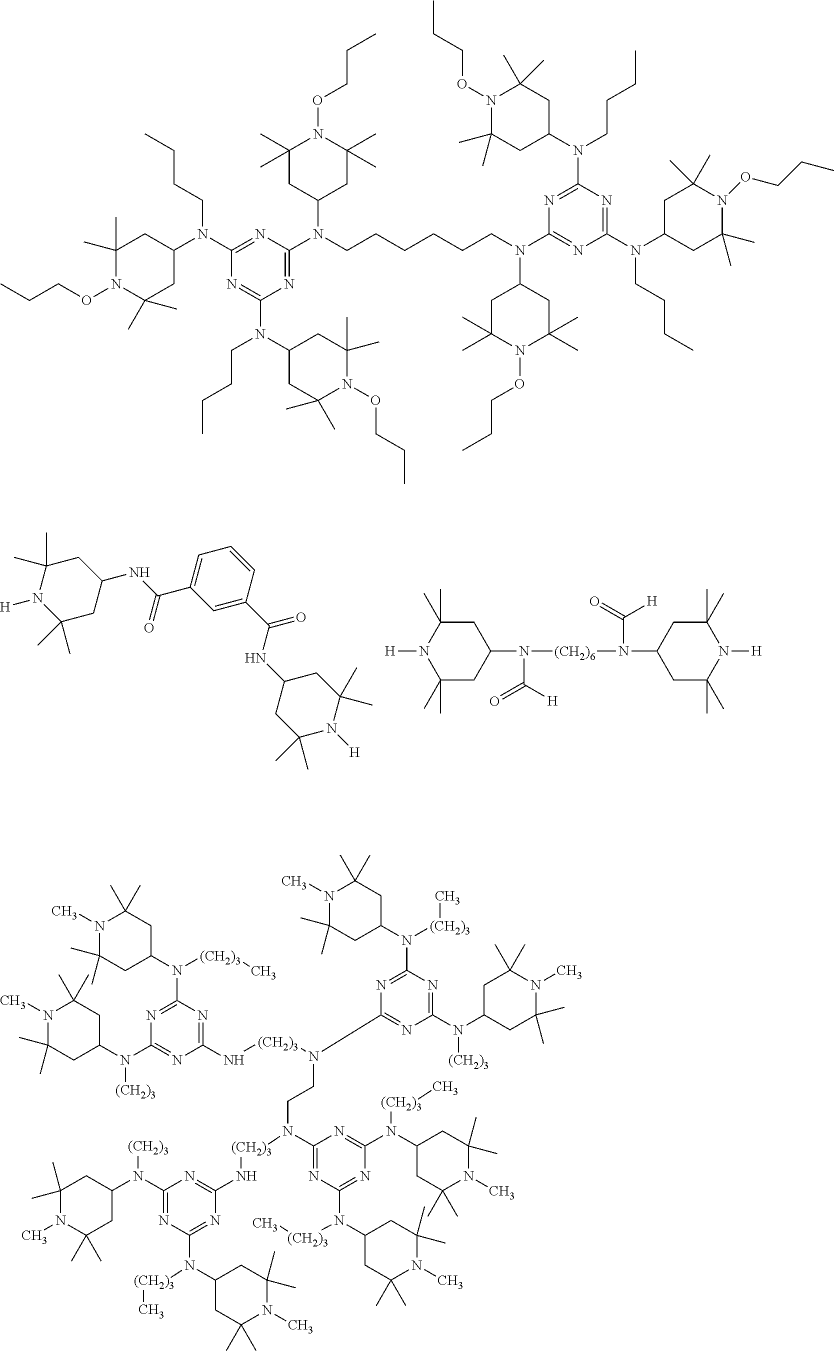

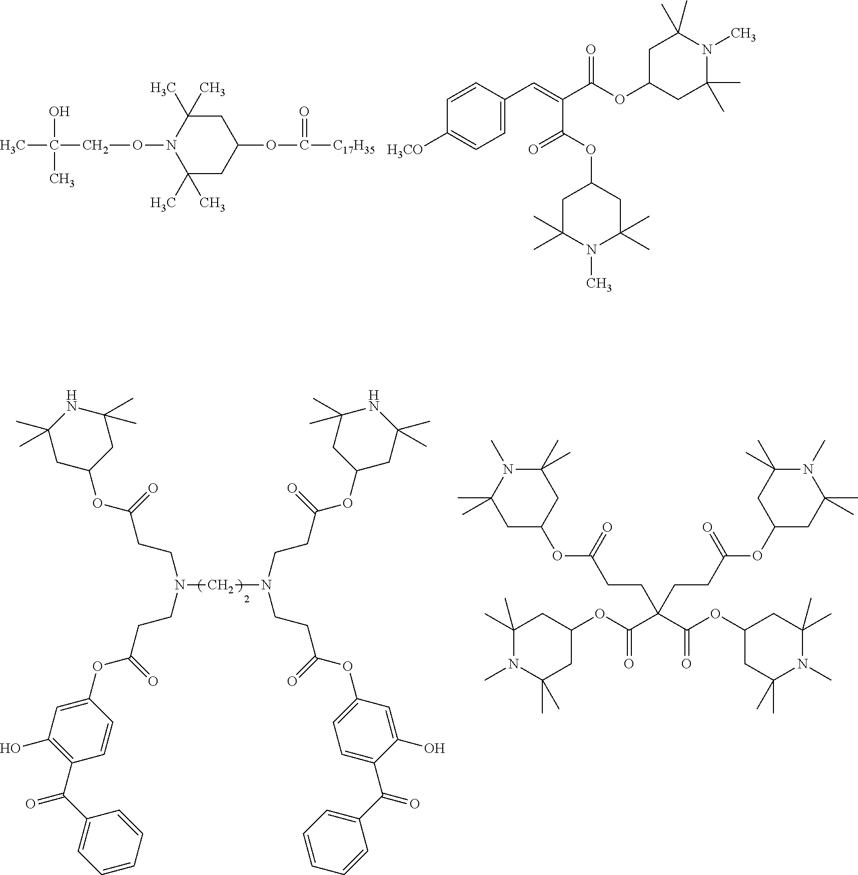

Preferred oligomeric and polymeric hindered amines have the following structures:

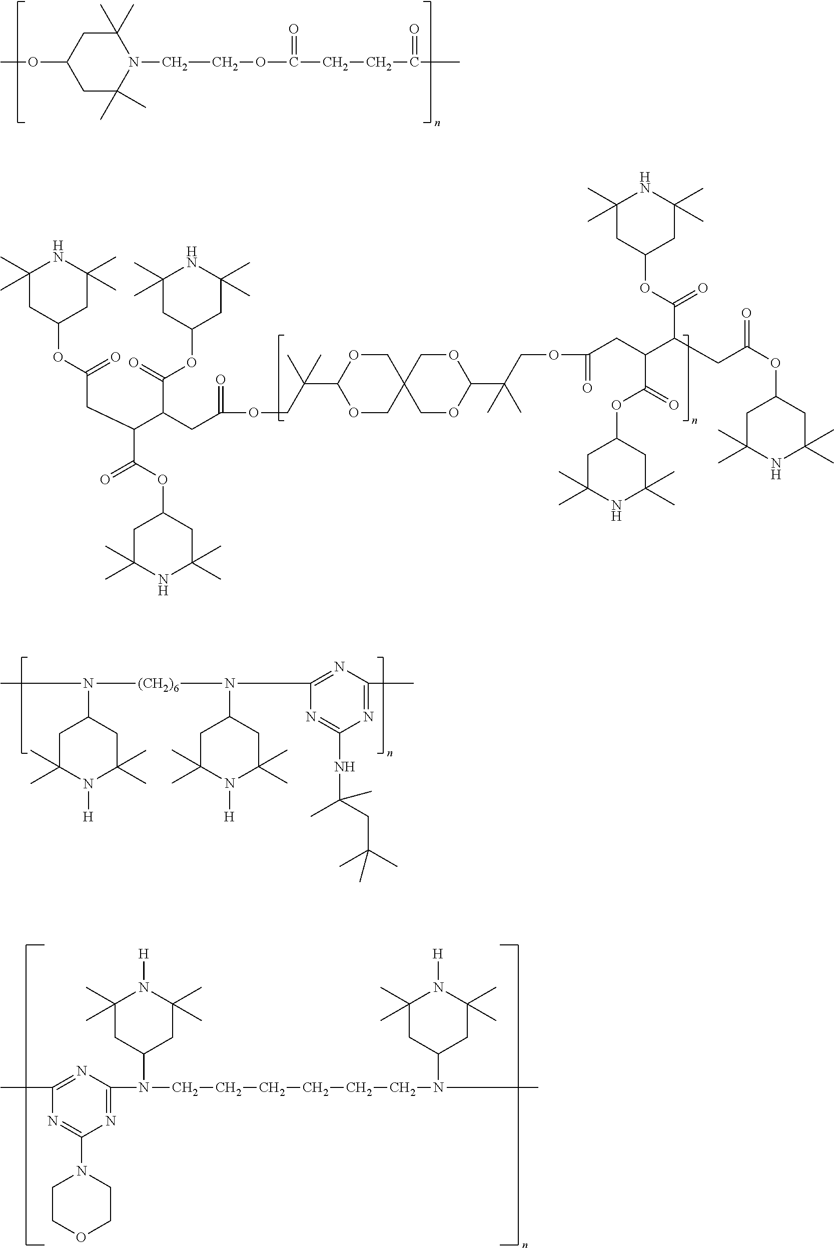

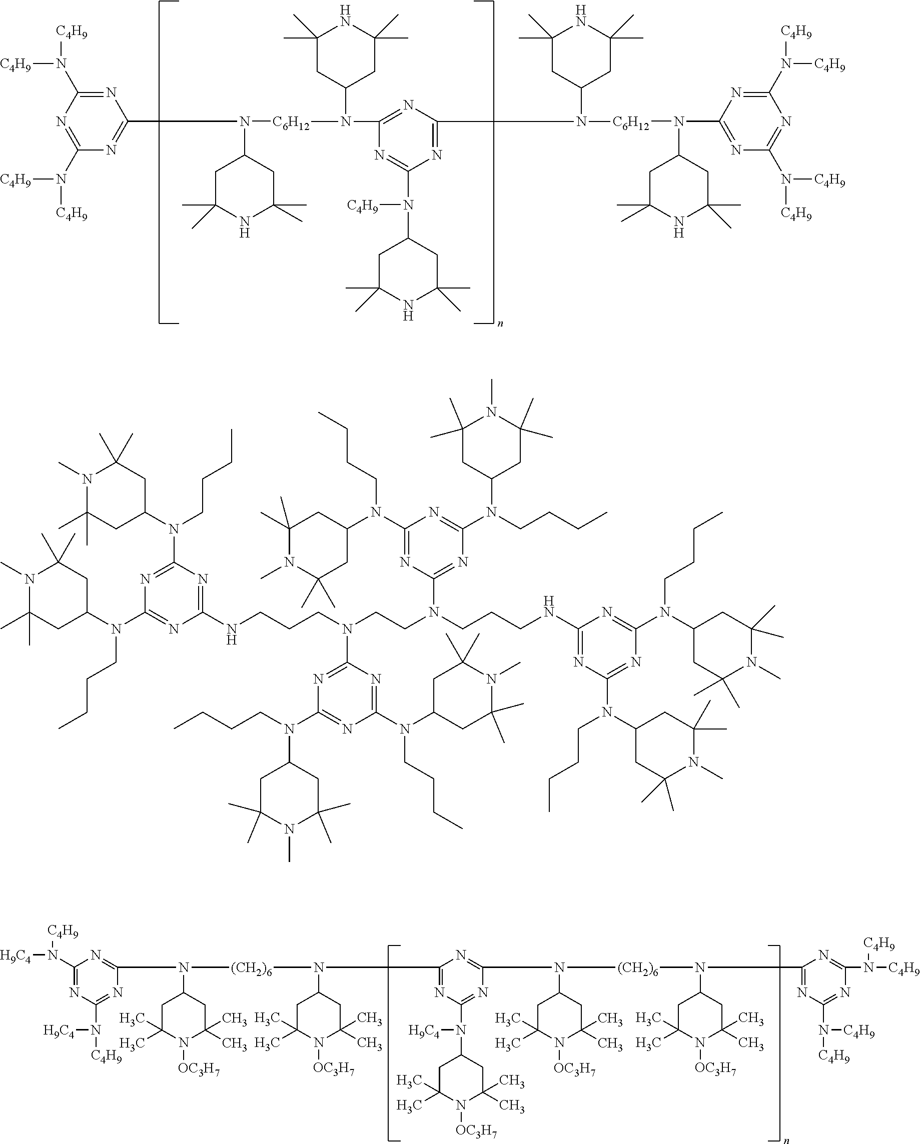

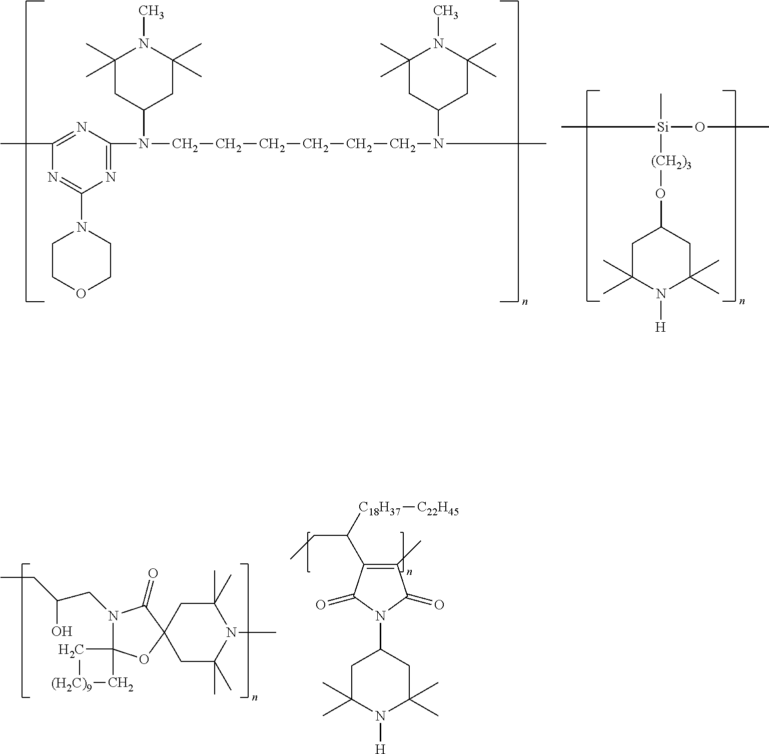

In the above-named compounds, n respectively means 3 to 100.

A further suitable light stabilizer is Hostanox NOW (manufacturer: Clariant SE) having the following general structure:

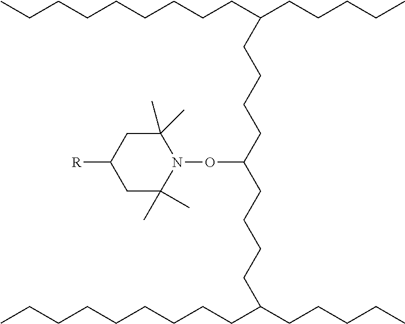

where R is —O—C(O)—C₁₅H₃₁ or —O—C(O)—C₁₇H₃₅.

Suitable dispersion agents are, for example:

polyacrylates, e.g. copolymers with long-chain side groups, polyacrylate block copolymers, alkylamides: e.g. N,N′-1,2-ethanediylbisoctadecanamide sorbitan esters, e.g. monostearylsorbitan esters, titanates and zirconates, reactive copolymers with functional groups, e.g. polypropylene-co-acrylic acid, polypropylene-co-maleic acid anhydride, polyethylene-co-glycidylmethacrylate, polystyrene-alt-maleic acid anhydride polysiloxanes: e.g. dimethylsilanediol-ethylene oxide copolymers, polyphenylsiloxane copolymers, amphiphilic copolymers: e.g. polyethylene block polyethylene oxide, dendrimers, e.g. dendrimers containing hydroxyl groups.

Suitable antinucleation agents are azine dyes such as nigrosin.

Suitable flame retardant agents are, in particular
a) inorganic flame retardant agents such as e.g. Al(OH)₃, Mg(OH)₂, AlO(OH), MgCO₃, sheet silicates such as montmorillonite or sepiolite, unmodified or organically modified double salts such as Mg—Al silicates, POSS (polyhedral oligomeric silsesquioxanes) compounds, huntite hydromagnesite or halloysite and Sb₂O₃, Sb₂O₅, MoO₃, zinc stannate, zinc hydroxystannate,
b) flameproofing agents containing nitrogen such as melamine, melem, melam, melon, melamine derivatives, melamine condensation products or melamine salts, benzoguanamine, polyisocyanurates, allantoin, phosphacenes, in particular melamine cyanurate, melamine phosphate, dimelamine phosphate, melamine pyrophosphate, melamine polyphosphate, melamine metal phosphates such as melamine aluminum phosphate, melamine zinc phosphate, melamine magnesium phosphate, and the corresponding pyrophosphates and polyphosphates, poly-[2,4-(piperazine-1,4-yl)-6-(morpholine-4-yl)-1,3,5-triazine], ammonium polyphosphate, melamine borate, melamine hydrobromide,
c) radical formers such as alkoxyamines, hydroxylamine esters, azo compounds, triazine compounds, disulfides, polysulfides, thiols, thiuram sulfides, dithiocarbamates, mercaptobenzthiazoles, sulfene amides, sulfene imides,
d) dicumyl or polycumyl, hydroxyimides and their derivatives such as hydroxyimide esters or hydroxyimide ethers,
e) flame retardant agents containing phosphorus such as red phosphorus, phosphates such as resorcin diphosphate, bisphenol-A-diphosphate, and their oligomers, triphenylphosphate, ethylene diamine diphosphate, phosphinates such as salts of hypophosphorous acid and their derivatives such as alkylphosphinate salts, e.g. orsindiphosphate diethylphosphinate aluminum or diethylphosphinate-zinc or aluminum phosphinate, aluminum phosphite, aluminum phosphonate, phosphonate esters, oligomer and polymer derivatives of the methane phosphonic acid, 9,10-dihydro-9-oxa-10-phosphorylphenanthrene-10-oxide (DOPO) and their substituted compounds,
f) halogenated flameproofing agents based on chlorine and bromine such as polybrominated diphenyl oxides such as decabromodiphenyloxide, tris(3-bromo-2,2-bis(bromomethyl)propyl-phosphate, tris(tribromoneopentyl)phosphate, tetrabromophthalic acid, 1,2-bis(tribromophenoxy)ethane, hexabromocyclododecane, brominated diphenylethane, tris-(2,3-dibrompropyl)isocyanurate, ethylene-bis-(tetrabromophthalimide), tetrabromo-bisphenol A, brominated polystyrene, brominated polybutadiene or polystyrene brominated polybutadiene copolymers, brominated polyphenylene ether, brominated epoxy resin, polypentabromobenzylacrylate, optionally in combination with Sb₂O₃ and/or Sb₂O₅,
g) borates such as zinc borate or calcium borate, optionally on a carrier material such as silica.
h) sulfur-comprising compounds, such as e.g. elemental sulphur, disulfides and polysulfides, thiuram sulfide, dithiocarbamates, mercaptobenzothiazole and sulfenamides,
i) anti-drip agents, such as e.g. polytetrafluoroethylene,
j) silicon-comprising compounds, such as polyphenyl siloxanes,
k) carbon modifications, such as carbon nanotubes (CNTs), expandable graphite or graphene
l) and combinations or mixtures thereof.

The following compounds are very particularly preferred flame retardant agents: Al(OH)₃, Mg(OH)₂,

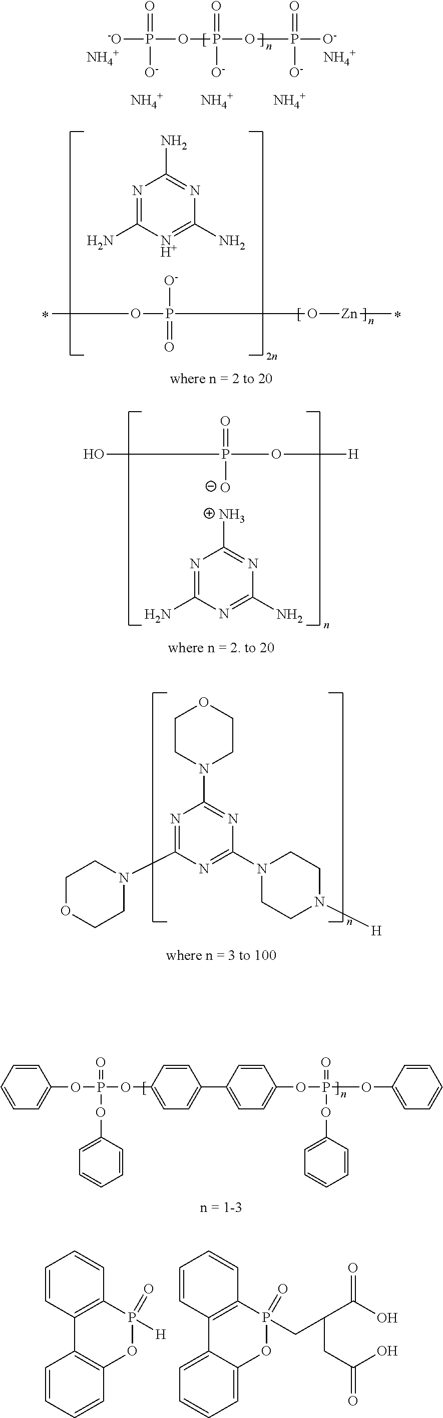

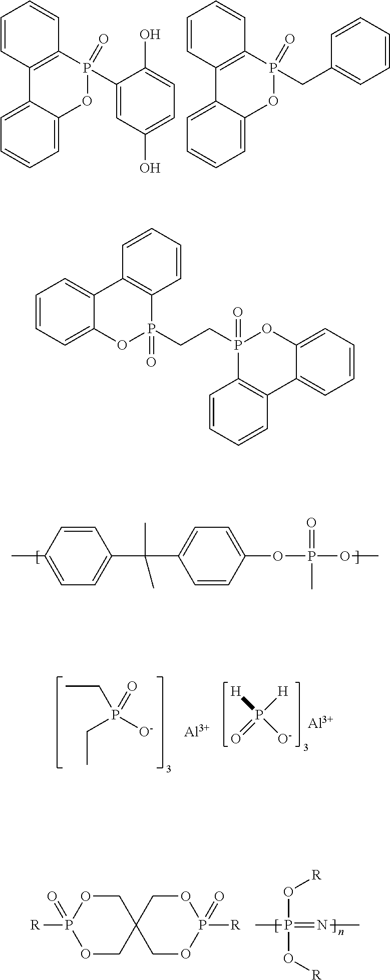

where in each case R=alkyl, phenyl, and where n=3 to 20

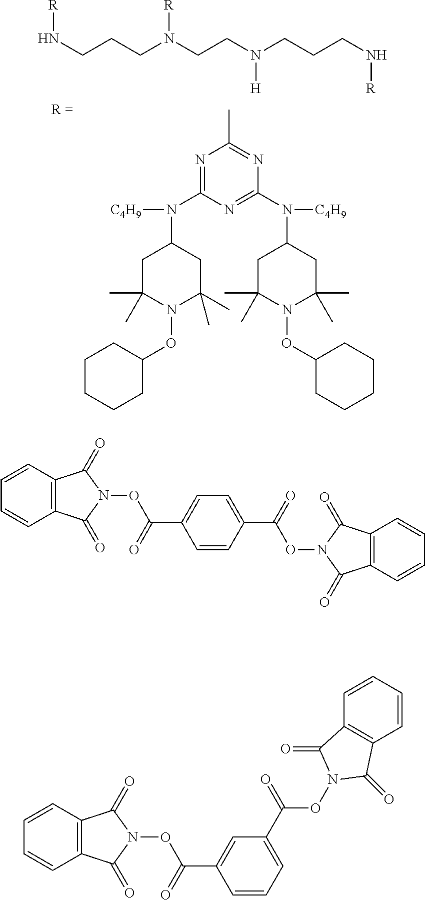

Suitable plasticizers are, for example, phthalic acid esters, adipic acid esters, esters of citric acid, esters of 1,2-cyclohexane dicarboxylic acid, tremolitic acid esters, isosorbide esters, phosphate esters, epoxides such as epoxidized soy bean oil, or aliphatic polyesters.

Suitable slip agents and processing aids are, for example, polyethylene waxes, polypropylene waxes, salts of fatty acids such as calcium stearate, zinc stearate, or salts of montane waxes, amide waxes such as erucic acid amide or oleic acid amides, fluoropolymers, silicones, or neoalkoxytitanates and zirconates.

Suitable pigments can be of an inorganic or organic nature. Inorganic pigments are, for example, titanium dioxide, zinc oxide, zinc sulfide, iron oxide, ultramarine, black carbon; organic pigments are, for example, anthraquinones, anthanthrones, benzimidazolones, chinacridones, diketoptyrrolopyrrols, dioxazines, inanthrones, isoindolines, azo compounds, perylenes, phthalocyanines or pyranthrones. Further suitable pigments include effect pigments on a metal base or pearl gloss pigments on a metal oxide base.

Suitable optical brighteners are, for example, bis-benzoxazoles, phenylcumarines, or bis(styryl)biphenyls and in particular optical brighteners of the formulas:

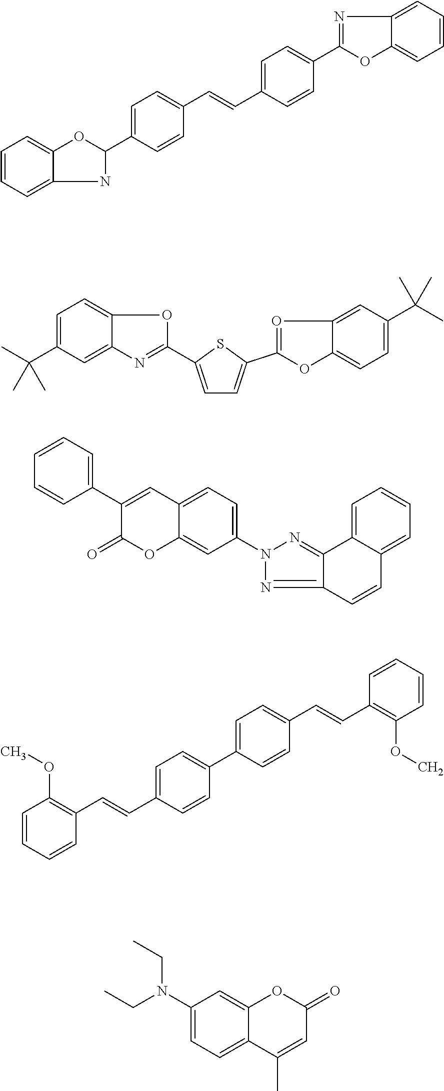

Suitable filler deactivators are, for example, polysiloxanes, polyacrylates, in particular block copolymers such as polymethacrylic acid polyalkyene oxide or polyglycidyl(meth)acrylates and their copolymers, e.g. with styrene and epoxides of e.g. the following structures:

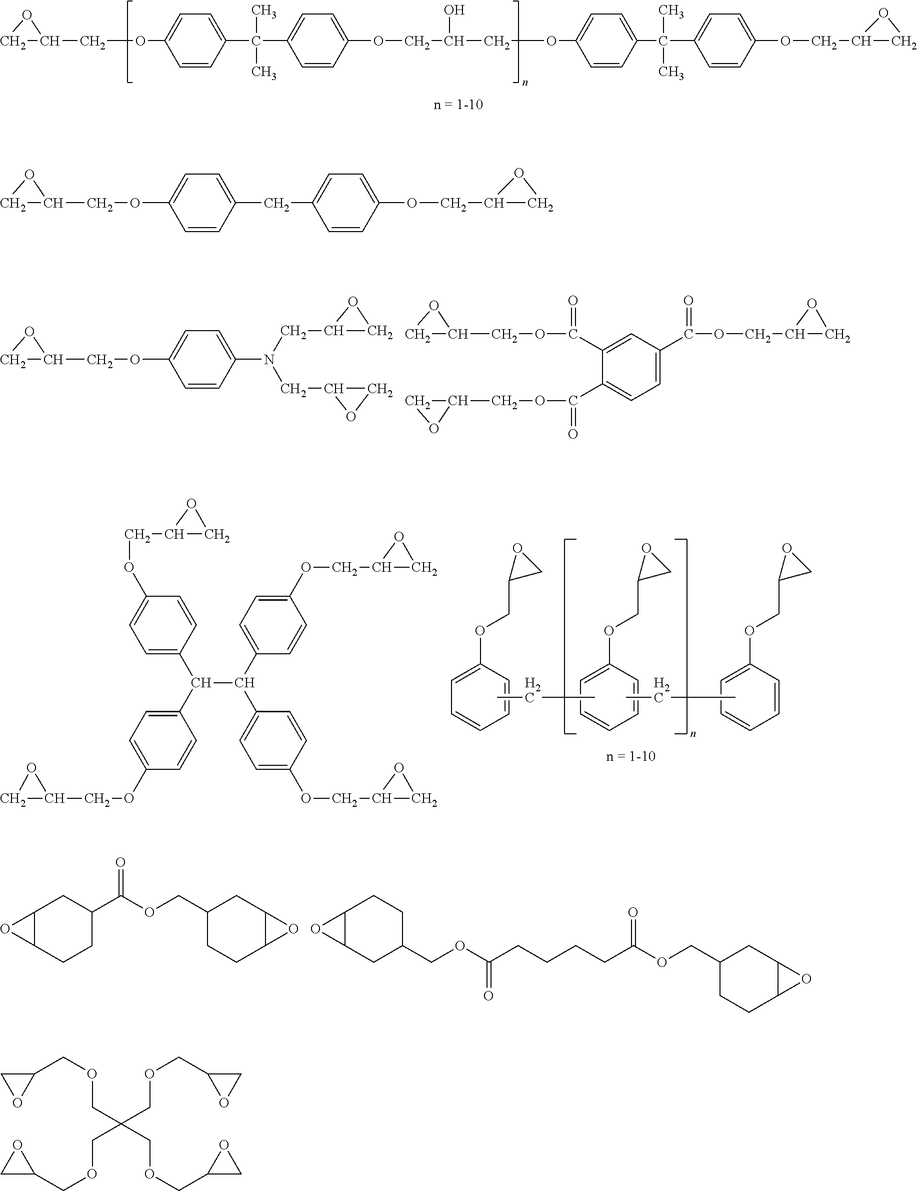

Suitable antistatic agents are, for example, ethoxylated alkylamines, fatty acid esters, alkylsulfonates, and polymers such as polyetheramides.

Suitable antiozonants are the above-mentioned amines such as N,N′-di-isopropyl-p-phenylenediamine, N,N′-di-sec-butyl-p-phenylenediamine, N,N′-bis(1,4-dimethylpentyl)-p-phenylenediamine, N,N′-dicyclohexyl-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine

Suitable rheology modifications, e.g. for the preparation of controlled rheology polypropylene (CR-PP) are, for example, peroxides, alkoxyaminoesters, oxymide sulfonic acid esters, and in particular the following structures:

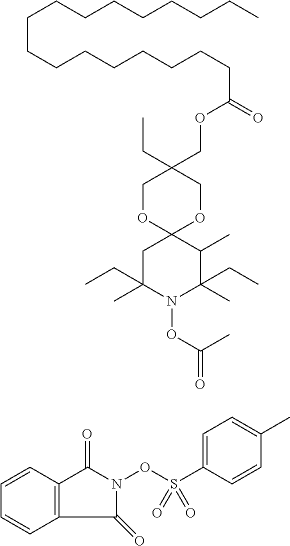

Suitable nucleation agents are, talcum, alkali, or alkaline earth salts of mono- and polyfunctional carboxylic acids such as benzoic acid, succinic acid, adipic acid, e.g. sodium benzoate, zinc glycerolate, aluminiumhydroxy-bis(4-tert-butyl)benzoate, 2,2′-methylene-bis-(4,6-di-tert-butylphenyl)phosphate, and trisamides and diamides such as trimesic acid tricyclohexylamide, trimesic acid tri(4-methylcyclohexylamide), trimesic acid tri(tert-butylamide), N,N′,N″-1,3,5 benzoltriyltris(2,2-dimethyl-propanamide) or 2,6-naphthalene dicarboxylic acid cyclohexylamide.

Suitable additives for the linear molecular weight structure of polycondensation polymers (chain extenders) are diepoxides, bis-oxazonlines, bis-oxazolones, bis-oxazines, diisoscyanates, dianhydrides, bis-acyllactams, bis-maleimides, dicyanates, carbodiimides. Further suitable chain extenders are polymeric compounds, such as e.g. polystyrene-polyacrylate-polyglycidyl (meth)acrylate copolymers, polystyrene-maleic anhydride copolymers and polyethylene-maleic anhydride copolymers.

Suitable additives for increasing the electrical conductivity are, for example, the antistatic agents mentioned, carbon black and carbon compounds like carbon nanotubes and graphene, metal powder, such as e.g. copper powder, and conductive polymers, such as e.g. polypyrroles, polyanilines and polythiophenes. Suitable additives to increase thermal conductivity are, for example, aluminum nitrides and boron nitrides.

Suitable infrared-active additives are, for example, aluminum silicates or dyestuffs such as phthalocyanines or anthraquinones.

Suitable demolding aids are, for example, silicones, soaps, and waxes, such as montan waxes.

The additives in accordance with the invention can furthermore be used for the stabilization of oils, fats, and chemical products. If the organic materials are oils and fats, they can be on the basis of mineral oils, vegetable fats, or animal fats, or also oils, fats, or waxes on the basis of e.g. synthetic esters. Vegetable oils and fats are, for example, palm oil, olive oil, rape oil, linseed oil, soybean oil, sunflower oil, castor oil; animal fats are, for example, fish oils or suet. The compounds in accordance with the invention can furthermore be used as stabilizers of lubricants, hydraulic oils, engine oils, turbine oils, transmission oils, metal machining fluids, or as lubricating greases. These mineral or synthetic lubricants are primarily based on hydrocarbons. With chemical products it is e.g. the stabilization of polyols in polyurethane manufacture or of monomers such as styrene, acrylic esters or methacrylic esters for transport and storage.

The incorporation of the additives described above and optionally of the additional additives into the plastic takes place by typical processing methods, with the polymers being melted and being mixed with the additive composition in accordance with the invention and the optionally further additives, preferably by mixers, kneaders, and extruders. Extruders such as single screw extruders, twin screw extruders, planetary gear extruders, ring extruders, and co-kneaders that are preferably equipped with a vacuum degassing are preferred as processing machines. The processing can take place here under air or, optionally, under inert gas conditions.

The additive compositions in accordance with the invention can furthermore be prepared and incorporated in a polymer in the form of so-called master batches or concentrates that, for example, include 10-90% of the stabilizers or compositions in accordance with the invention.

In particular the additives described above can be considered as possible additives. Secondary antioxidants are, for example, particularly preferred here, in particular selected from the group consisting of phosphites, phosphonites, and thiols, costabilizers, selected from the group consisting of polyols, acid scavengers, and sterically hindered amines or mixtures and combinations thereof.

It is particularly preferred here if the at least one additive is contained or is added in an amount of 0.01 to 80 wt %, preferably of 0.01 to 9.99 wt %, further preferably of 0.01 to 4.98 wt %, particularly preferably of 0.02 to 2.00 wt % with respect to the totality of the at least one compound in accordance with formula I of the organic material and of the at least one additive.

The present invention additionally relates to an organic material, in particular a plastic composition, comprising at least one compound in accordance with general formula I or a mixture of a plurality of compounds in accordance with general formula I as the stabilizer

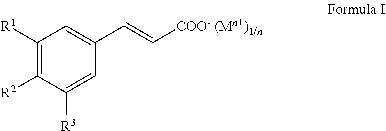

where R¹, R², R³ M, and n are defined as above.

All the advantageous embodiments described in connection with the use in accordance with the invention equally apply without restriction to the organic material.

In a preferred embodiment, the organic material has the following composition:

0.01 to 10.00 wt %, preferably 0.01 to 7.50 wt %, further preferably of 0.02 to 5.00 wt %, particularly preferably of 0.050 to 2.00 wt %, of a compound in accordance with the general formula I or, in the case of a mixture of a plurality of compounds in accordance with the general formula I, the totality of all the compound in accordance with the general formula I
99.99 to 10.00, preferably 99.99 to 90.00 wt %, preferably 99.89 to 95.00 wt %, particularly preferably 99.90 to 98.00 wt %, of at least one organic material, preferably selected from the group consisting of plastics, coatings, lubricants, hydraulic oils, engine oils, turbine oils, transmission oils, metal machining fluids, chemicals, or monomers, and

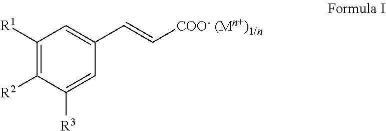

0 to 80.00 wt %, preferably 0 to 9.99 wt %, further preferably 0.01 to 4.98 wt %, particularly preferably 0.02 to 2.00 wt %, of at least one additive,
where the components add up to 100%.

The invention additionally relates to a method of stabilizing organic materials, in particular against oxidative, thermal and/or actinic degradation, in which a compound or a plurality of compounds in accordance with general formula I

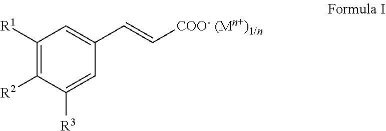

where R¹, R², R³ and M and n are defined as above is incorporated into the organic material.

The present invention additionally relates to compounds in accordance with the general formula I

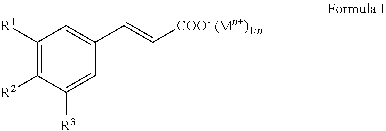

where
R¹, R² and R³ are each selected independently of one another from the group consisting of hydroxy, linear or branched alkoxy group having 1 to 6 carbon atoms, and hydrogen, with the proviso that at least one of the residues R¹, R² and R³ is a hydroxy residue, and
M is aluminum, and
n is 3.

A further aspect of the present invention relates to a stabilizer composition comprising or consisting of
a) a compound or mixtures of a plurality of compounds in accordance with general formula I

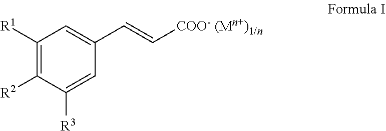

where R¹, R², R³ M, and n are defined as above (component A) and
b) at least one secondary antioxidant selected from the group consisting of phosphites, phosphonites, or thiols, or at least one costabilizer selected from the group consisting of polyols, acid scavengers, or sterically hindered amines, and mixtures and combinations thereof (component B).

It is in particular of advantage here if component A and component B are present in the stabilizer composition in a weight ratio of 100:1 to 1:100, preferably 10:1 to 1:10, particularly preferably of 4:1 to 1:4.

The present invention will be illuminated in more detail with reference to the following embodiments without restricting the invention to the specific embodiments.

### EMBODIMENTS

A) Preparation of the Hydroxycinnamic Acid Salts in Accordance with the Invention

### A1) Synthesis of the Sodium Ferulate (NaFa)

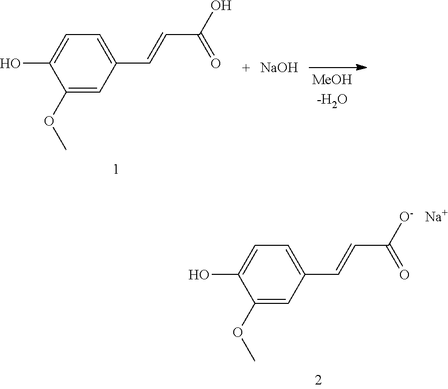

12.00 g (1.00 eq., 61.80 mmol) ferulic acid (1) are first dissolved in 620 mL methanol in a glass beaker. Subsequently, 2.47 g (1.00 eq., 61.80 mmol) sodium hydroxide are dissolved in 60 mL distilled water and the resulting NaOH solution is dripped into the ferulic acid solution via a drip funnel. The slightly yellow solution is added to 1.5 l acetone after the addition has ended. The precipitated yellow precipitation is filtered, is washed 3 times with 200 mL acetone in each case, and is finally dried in the vacuum drying cabinet at 80° for two days. 10.51 g of a yellow fine-powdered precipitate are obtained. The yield amounts to 78.66%.

### A2) Synthesis of the Aluminum Ferulate (AlFa)

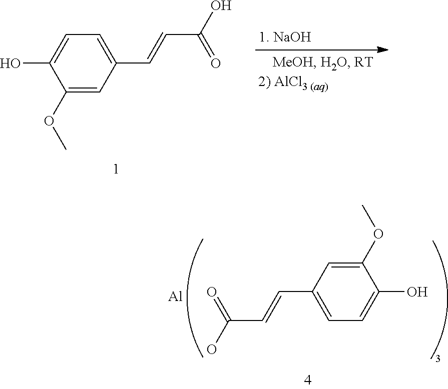

11.18 g (3.07 eq., 57.57 mmol) ferulic acid are first dissolved in 50 mL methanol. 57 mL of a 1 M NaOH solution is added to the yellow solution. After the solution has been stirred for half an hour, a solution of 2.5 g (1.00 eq., 18.75 mmol) aluminum trichloride and 2.8 mL distilled water are dripped in via a drip funnel. A white precipitate is immediately deposited here that is stirred for a further hour and then filtered. After the washing three times with 200 mL distilled water and 200 mL acetone respectively and after the drying in the vacuum drying cabinet at 80° C. overnight, 5.32 g of a white coarse grain solid are obtained.

### A3) Synthesis of the Magnesium Ferulate (MgFa)

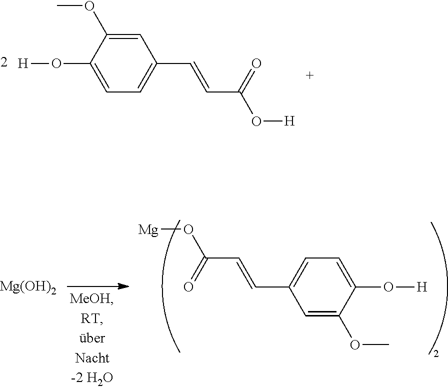

2.5 g (1.00 eq., 42.86 mmol) magnesium hydroxide are first suspended in 600 mL distilled water in a glass beaker. Subsequently, 16.91 g (2.03 eq., 87.08 mmol) ferulic acid is dissolved while heating into 250 mL methanol and the resulting slightly yellow solution is added to the magnesium hydroxide suspension. The suspension is stirred at room temperature overnight, with the magnesium hydroxide entering into solution. The resulting slightly yellow solution is added to 1.5 l acetone. The precipitated yellow precipitation is filtered, is washed 3 times with 200 mL acetone in each case, and is finally dried in the vacuum drying cabinet at 800 for two days. 2.80 g of a yellow fine-powdered precipitate are obtained. The yield amounts to 15.90%.

### A4) Synthesis of the Calcium Ferulate (CaFa)

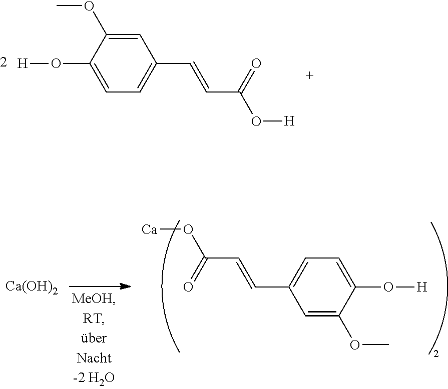

2.00 g (1.00 eq., 26.99 mmol) calcium hydroxide are first suspended in 100 mL distilled water in a glass beaker. Subsequently, 10.48 g (2.00 eq., 53.97 mmol) ferulic acid is dissolved while heating into 100 mL methanol and the resulting slightly yellow solution is added to the calcium hydroxide suspension. The suspension is stirred at room temperature overnight, with the calcium hydroxide entering into solution. At the same time, a silvery residue remains that is filtered. The filtrate is added to 1.5 l acetone. The precipitated yellow precipitation is filtered, is washed 3 times with 200 mL acetone in each case, and is finally dried in the vacuum drying cabinet at 80° for two days. 7.42 g of a yellow fine-powdered precipitate is obtained. The yield amounts to 64.47%.

### B) Application Check

A commercial polypropylene (Molen HP 501B, Lyondell Basell Industries) was homogenized in a powder-powder mixture with the stabilizers or stabilizer mixtures specified in the tables and was conducted in a circuit in a twin screw microextruder (MC 5, manufacturer DSM) at 200° C. and at 200 revolutions per minute for 30 minutes and the reduction of the force was recorded to check the effect of the stabilizers in accordance with the invention. The force is a direct measure for the molecular weight of polypropylene; the smaller the reduction, the higher the stabilization effect.

TABLE 1
Stabilization of polypropylene

| | | Residual force |
|---|---|---|
| | | after 10/20/30 |
| | Additive | minutes [%] |
| Comparison example 1 | Without additive | 63/39/26 |
| Example 1 in accordance | 0.5% sodium ferulate | 76/59/44 |
| with the invention | | |
| Example 2 in accordance | % aluminum ferulate | 75/61/51 |
| with the invention | | |

TABLE 2
Stabilization of polypropylene

| | | Residual force |
|---|---|---|
| | | after 10/20/30 |
| | Additives | minutes [%] |
| Example 3 in accordance | 0.25% sodium ferulate | 86/81/75 |
| with the invention | and 0.25% erythritol | |
| Example 4 in accordance | 0.25% aluminum ferulate | 85/76/67 |
| with the invention | and 0.25% erythritol | |
| Example 5 in accordance | 0.25% calcium ferulate | 73/55/40 |
| with the invention | and 0.25% erythritol | |

TABLE 3
Stabilization of polypropylene

| | | Residual force |
|---|---|---|
| | | after 10/20/30 |
| | Additives | minutes [%] |
| Example 6 in accordance | 0.25% sodium ferulate | 90/81/70 |
| with the invention | and 0.25% methionine | |

TABLE 3
Stabilization of polypropylene

| | | Residual force |
|---|---|---|
| | | after 10/20/30 |
| | Additives | minutes [%] |
| Example 7 in accordance | 0.25% sodium ferulate | 86/73/66 |
| with the invention | and 0.25% DSDTP | |
| Example 8 in accordance | 0.25% sodium ferulate | 86/73/60 |
| with the invention | and 0.25% phosphite | |

DSDTP = distearyl thiodipropionate
Phosphit = Tris-(2,4-di-tert-butylphenyl)phosphit

The additives in accordance with the invention display a considerable stabilization effect since a smaller reduction of the polymer takes place over the trial period.

### C) Oxidation Induction Time (OIT)

The oxidation induction time is a standardized test that is carried out in a differential calorimeter. This method permits a determination of the thermal stability of the material to be inspected. The time between the melting and the start of the degradation under isothermal conditions (220° C. here) is determined here. A nitrogen atmosphere is present here up to the melting of the material to be checked; synthetic air is subsequently supplied. In Table 4, the additive combinations incorporated and checked in commercial polypropylene (Moplen HP 500N, Lyondell Basell Industries) by means of a corotating twin screw laboratory extruder (Process 11, Thermo Fisher Scientific) at an extrusion temperature of 200° C. are summarized.

TABLE 4
Oxidation induction time of the compounds
incorporated in polypropylene.

| | OIT₁ | OIT₂ | O̅I̅T̅ |
|---|---|---|---|
| Additive combinations | [min] | [min] | [min] |
| Comparison example | 5.21 | 4.59 | 4.90 |
| Without additive | | | |
| Example in accordance | 18.56 | 24.92 | 21.74 |
| with the invention 0.50% | | | |
| sodium ferulate | | | |

It is shown that a considerable increase of the oxidative stability of polypropylene is achieved by the addition of 0.5% sodium ferulate.

1-17. (canceled)

18. A method for stabilizing an organic material comprising combining the organic material with a compound or a mixture of compounds in accordance with general formula I 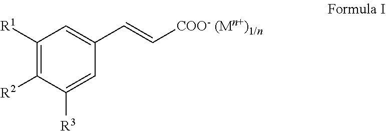
wherein
R¹, R², and R³ are each selected independently of one another from the group consisting of hydroxy, linear or branched alkoxy group having 1 to 6 carbon atoms, and hydrogen, with the proviso that at least one of the residues R¹, R² and R³ is a hydroxy residue,
M is selected from the group consisting of metals, and
n is a integer from 1 to 4.

19. The method of claim 18, wherein the compound stabilizes the organic material against oxidative, thermal and/or actinic degradation.

20. The method of claim 18, wherein the organic material is selected from plastics, coatings, lubricants, hydraulic oils, engine oils, turbine oils, transmission oils, metal machining liquids, chemicals, and monomers.

21. The method of claim 18, wherein
the residues R¹, R², and R³ each represents a hydroxy residue,
two of the residues R¹, R², and R³ represent a hydroxy residue and one of the residues R¹, R², and R³ represents hydrogen or a linear or branched alkoxy group having 1 to 6 carbon atoms,
one of the residues R¹, R², and R³ represents a hydroxy residue and two of the residues R¹, R², and R³ represent a linear or branched alkoxy group having 1 to 6 carbon atoms, or
one each of the residues R¹, R² and R³ represents a hydroxy residue, a linear or branched alkoxy group having 1 to 6 carbon atoms, and hydrogen.

22. The method of claim 18, wherein the compound in accordance with general formula I is selected from the group consisting of the following compounds:

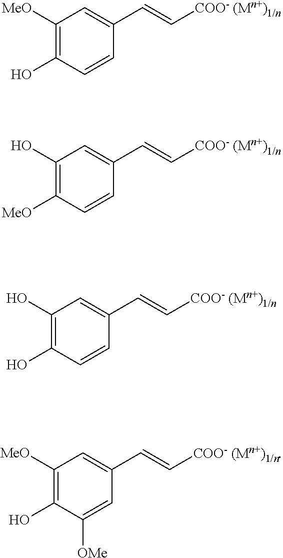

23. The method of claim 18, wherein M is selected from the group consisting of alkali metals, alkaline earth metals, aluminum, and zinc.

24. The method of claim 18, wherein the compound in accordance with general formula I or, in the case of a mixture of a plurality of compounds in accordance with general formula I, the totality of all the compounds in accordance with general formula I is included in the organic material at a weight proportion of 0.01 to 10.00 wt. %.

25. The method of claim 18, wherein the plastic is selected from the group consisting of
a) polyethylene, metallocene PE (m-PE), polypropylene, polyisobutylene, poly-4-methyl-pentene-1, polybutadiene, polyisoprene, polycyclooctene, polyalkylene carbon monoxide copolymers, polypropylene polyethylene (EP) copolymer, EPM or EPDM copolymer, ethylene vinyl acetate (EVA) copolymer, ethylene acrylic ester copolymer, ethylene acrylic acid copolymer and its salts, ethylene acrylic acid glycidyl(meth)acrylate terpolymer, polypropylene graft maleic acid anhydride, polypropylene graft acrylic acid, polyethylene graft acrylic acid, polyethylene polybutyl acrylate graft maleic acid anhydride, and blends thereof,
b) polystyrene, polymethylstyrene, poly-alpha-methylstyrene, polyvinyl naphthalene, polyvinyl biphenyl, polyvinyl toluol, styrene butadiene (SB), styrene butadiene styrene (SBS), styrene ethylene butylene styrene (SEBS), styrene ethylene propylene styrene, styrene isoprene, styrene isoprene styrene (SIS), styrene butadiene acrylonitrile (ABS), styrene acrylonitrile (SAN), styrene acrylonitrile acrylate (ASA), styrene ethylene, styrene maleic acid anhydride polymers, graft copolymers of styrene on butadiene, graft copolymers of maleic acid anhydride on SBS or SEBS, graft copolymers of methylmethacrylte, styrene butadiene, and ABS (MABS), and hydrated polystyrene derivatives;
c) halogen-containing polymers selected from the group consisting of polyvinyl chloride (PVC), polychloroprene, and polyvinylidene chloride (PVDC), copolymers of vinyl chloride and vinylidene chloride or composed of vinyl chloride and vinyl acetate, chlorinated polyethylene, polyvinylidene fluoride, and epichlorohydrin homo and copolymer thereof,
d) polymers of unsaturated esters selected from the group consisting of polymethyl methacrylate (PMMA), polybutyl acrylate, polyauryl acrylate, poly stearyl acrylate, and polyglycidyl methacrylate, polyacrylonitrile, polyacrylamides, and polyacrylonitrile-poly alkyl acrylate;
e) polymers of unsaturated alcohols and derivatives selected from the group consisting of polyvinyl alcohol, polyvinyl acetate, polyvinyl butyrate, and poly allyl phthalate, and poly allyl melamine;
f) polyacetates;
g) polyphenylene oxides and blends with polystyrene or polyamides,
h) polymers of cyclic ethers;
i) polyurethanes of hydroxy terminated polyethers or polyesters and aromatic or aliphatic isocyanates, and polyureas;
j) polyamides selected from the group consisting of polyamide-6, 6.6, 6.10, 4.6, 4.10, 6.12, 10.10, 10.12, 12.12, polyamide 11, polyamide 12, partly or fully aromatic polyamides, blends of different polyamides, and blends of polyamides and polyolefins;
k) a polymer selected from the group consisting of polyamides, polyamide imides, polyether imides, polyester imides, poly(ether)ketones, polysulfones, polyether sulfones, polyaryl sulfones, polyphenylene sulfides, polybenzimide azoles, and polyhydantoines;
l) polyesters of aliphatic or aromatic dicarboxylic acids and diols or of hydroxy carboxylic acids;
m) polycarbonates, polyester carbonates, and blends;
n) cellulose derivatives,
o) epoxy resins,
p) phenol resins;
q) unsaturated polyester resins of unsaturated dicarboxylic acids and diols with vinyl compounds;
r) silicones; and
s) mixtures, combinations, or blends of two or more of the above-named polymers.

26. The method of claim 18, wherein the plastic comprises at least one further additive.

27. The method of claim 26, wherein the further additive is selected from the group consisting of primary and/or secondary antioxidants, UV absorbers, light stabilizers, stabilizers on a hydroxyl amine base, stabilizers on a benzofuranone base, nucleating agents, toughness improvers, plasticizers, mold lubricants, rheological modifiers, chain extenders, processing aids, pigments, dyes, optical brighteners, antimicrobial active agents, antistatic agents, slip agents, anti-blocking agents, coupling agents, dispersing agents, compatibilizers, oxygen scavengers, acid scavengers, costabilizers, marking agents, and anti-fogging agents.

28. The method of claim 27, wherein the at least one additive is added or is included in an amount of 0.01 to 80 wt % with respect to the totality of the at least one compound in accordance with formula I of the organic material and of the at least one additive.

29. The method of claim 18, wherein the compound or mixtures of a plurality of compound in accordance with the general formula I is/are combined along with a costabilizer.

30. The method of claim 29, wherein the costabilizer is selected from the group consisting of polyols, hyper branched polymers/oligomers or dendrimers having alcohol groups, threitol, erythritol, galactitol, mannitol, ribitol, sorbitol, xylitol, arabitol, isomaltol, lactitol, maltitol, altritol, iditol, maltotritol, and hydrated oligo- and polysaccharides having polyol end groups, and mixtures thereof.

31. A composition comprising an organic material in combination with at least one compound in accordance with general formula I or a mixture of a plurality of compounds in accordance with general formula I as a stabilizer 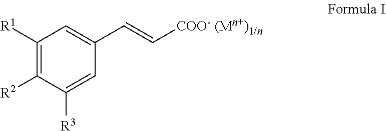
wherein
R¹, R², and R³ are each selected independently of one another from the group consisting of hydroxy, linear or branched alkoxy group having 1 to 6 carbon atoms, and hydrogen, with the proviso that at least one of the residues R¹, R² and R³ is a hydroxy residue,
M is selected from the group consisting of metals, and
n is a integer from 1 to 4.

32. The composition of claim 31, having the following composition
0.01 to 10.00 wt % of a compound in accordance with the general formula I or, in the case of a mixture of a plurality of compounds in accordance with the general formula I, the totality of all of the compounds in accordance with the general formula I,
99.99 to 10.00 wt % of at least one organic material, and
0 to 80.00 wt % of at least one additive,
wherein the components add up to 100%.

33. The composition of claim 32, wherein the at least one additive is selected from the group consisting of primary and/or secondary antioxidants, UV absorbers, light stabilizers, stabilizers on a hydroxyl amine base, stabilizers on a benzofuranone base, nucleating agents, toughness improvers, plasticizers, mold lubricants, rheological modifiers, chain extenders, processing aids, pigments, dyes, optical brighteners, antimicrobial active agents, antistatic agents, slip agents, anti-blocking agents, coupling agents, dispersing agents, compatibilizers, oxygen scavengers, acid scavengers, costabilizers, marking agents, and anti-fogging agents.

34. A compound in accordance with the general formula I 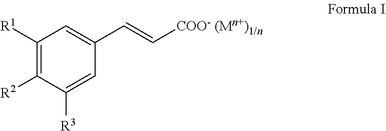
wherein
R¹, R², and R³ are each selected independently of one another from the group consisting of hydroxy, linear or branched alkoxy groups having 1 to 6 carbon atoms, and hydrogen, with the proviso that at least one of the residues R¹, R², and R³ is a hydroxy residue, and
M is aluminum, and
n is 3.

35. A stabilizer composition comprising
a) a compound or mixtures of a plurality of compounds in accordance with claim 34; and
b) at least one secondary antioxidant selected from the group consisting of phosphites, phosphonites, and thiols, or at least one costabilizer selected from the group consisting of polyols, acid scavengers, sterically hindered amines, and mixtures and combinations thereof (component B).

36. The stabilizer composition of claim 35, wherein component A and component B are present in a weight ratio of 100:1 to 1:100, 10:1 to 1:10, or 4:1 to 1:4.
